(19) United States
(12) Patent Application Publication
(10) Pub. No.: US 2023/0149689 A1
(43) Pub. Date: May 18, 2023

(54) BREAKAWAY CONNECTOR

(71) Applicant: Site Saver, Inc., Fayetteville, AR (US)

(72) Inventors: Will Armstrong, Fayetteville, AR (US); Vance Clement, Fayetteville, AR (US); Spencer Jones, Fayetteville, AR (US)

(21) Appl. No.: 18/097,254

(22) Filed: Jan. 15, 2023

Related U.S. Application Data
(63) Continuation-in-part of application No. 17/884,391, filed on Aug. 9, 2022.
(60) Provisional application No. 63/231,020, filed on Aug. 9, 2021.

(51) Int. Cl. A61M 39/26

(52) U.S. Cl. CPC A61M 39/26; A61M 2039/1072

(57) ABSTRACT

A method of selecting a breakaway connector apparatus for attachment to a needle-free connector on an intravenous (IV) line is provided. The method includes providing a needle-free connector for attachment to a breakaway connector. The needle-free connector includes a push-out force associated with the needle-free connector. The method further includes identifying the push-out force imparted by the needle-free connector, and identifying a desired range of separation force associated with separating a first portion of the IV line from a second portion of the IV line. The method further includes selecting a breakaway connector device including a housing and a breakaway component that is configured to detach from the housing when a breakaway force is applied to the device, such that a sum of the breakaway force and the push-out force is within the desired range of separation force.

A portion of the disclosure of this patent document contains material that is subject to copyright protection. The copyright owner has no objection to the reproduction of the patent document or the patent disclosure, as it appears in the U.S. Patent and Trademark Office patent file or records, but otherwise reserves all copyright rights whatsoever.

### CROSS REFERENCES TO RELATED APPLICATION

This application is a continuation-in-part of U.S. Pat. Application 17/884,391 filed Aug. 9, 2022 entitled BREAKAWAY CONNECTOR, which is a non-provisional of U.S. Pat. Application No. 63/231,020 filed Aug. 9, 2021 entitled BREAKAWAY CONNECTOR, which are hereby incorporated by reference in their entirety.

### STATEMENT REGARDING FEDERALLY SPONSORED RESEARCH DEVELOPMENT

Not Applicable.

### BACKGROUND

The present invention relates to devices and methods for fluid line connectors for medical and veterinary applications. More particularly, the present invention relates to breakaway connectors for attachment to needle-free connectors on intravenous fluid lines.

Conventional devices and methods for breakaway fluid line connectors generally include two housings joined together. A fluid pathway through the connector is broken when the two housings separate, and a valve in each housing is operable to stop the flow of fluid on each side when a separation event occurs. Conventional devices and methods utilizing such a configuration are used for a variety of applications, including intravenous (IV) medical lines including a soft, flexible tube placed inside a vein, usually in the hand or arm. When a sufficient amount of tension is applied to the line, the connector will separate, and the valves on each component of the connector will block fluid flow to prevent leakage.

In many medical applications, and particularly in peripheral IV lines, healthcare providers are increasingly implementing needle-free connectors to protect clinicians and patients. Needle-free connectors provide an access port to an IV line that does not require a needle insertion for transfer of fluids. Instead, needle-free connectors include a patient-side luer fitting with a seal that may be opened or pierced by a corresponding injection-side luer fitting or syringe. The seal receives the injection-side fitting in a sealed engagement, thereby establishing a fluid flow path between the injection-side component and the needle-free connector on the patient-side tubing apparatus. The seal may include a septum-style seal, an accordion seal, a compressible sheath, a push seal, or any other suitable seal known in the art for a needle-free engagement.

Needle-free connectors allow for quick connection and disconnection without the need for needles to transfer fluids. When the injection-side fitting is removed from the needle-free connector, the seal on the needle-free connector closes automatically, thereby preventing leakage of any fluid from the patient-side tubing assembly. As such, the needle-free connector on the patient-side includes an available seal which operates as a check valve to allow fluid flow into the patient-side tubing when opened, but preventing outflow when closed. These types of needle-free connectors have been rigorously designed to protect from contamination and can be easily cleaned to sterilize the external interfacing element and ensure microbes cannot ingress into the fluid path.

As mentioned above, when a sufficient amount of tension is applied to the line, the connector will separate. In many medical applications, it may be desirable (or clinically required) that the connector separates within a clinically accepted range of tension. For example, in order to protect clinicians and patients, it may be paramount that the connector does not separate under too little tension (e.g., a typical or otherwise expected bodily movement, adjustment of the line, etc.). Similarly, it may be paramount that the connector does in fact separate under a reasonable amount of tension (e.g., a powerful movement that, should the connector remain intact, causes injury or other issues).

Conventional breakaway fluid line connectors with two valves (one on each component side of the breakaway mechanism) are generally not optimized for use with needle-free connectors because such configurations effectively include three valves when installed on (e.g., attached to, engaged with, etc.) a needle-free connector. The three valves include the injection-side valve on the connector, the outflow side valve on the connector and the seal on the needle-free connector. This type of configuration unnecessarily includes an intermediate valve and leads to a bulky and oversized assembly at an infusion site. As such, it is desirable to provide improvements to breakaway connector devices to make the engagement more efficient in size, scale and operation, while separating under a tensile force that is within a desirable range for patient and clinician safety.

What is needed are improvements in devices and methods for breakaway connectors for use with fluid applications, and particularly for use with needle-free connectors in IV lines in medical and veterinary applications.

### BRIEF DESCRIPTION OF THE FIGURES

FIG. 1 illustrates a perspective view of an embodiment of a breakaway connector apparatus in accordance with the present disclosure.

FIG. 2 illustrates a perspective cross-sectional view of the embodiment of a breakaway connector apparatus of FIG. 1 with the axially-moveable cannula in a disengaged position.

FIG. 3 illustrates a perspective cross-sectional view of the embodiment of a breakaway connector apparatus of FIG. 1 with the axially-moveable cannula in an engaged position.

FIG. 4 illustrates a perspective exploded view of an embodiment of a breakaway connector apparatus in accordance with the present disclosure positioned for installation on a needle-free connector.

FIG. 5 illustrates a perspective view of an embodiment of a breakaway connector apparatus in accordance with the present disclosure installed on a needle-free connector.

FIG. 6 illustrates a cross-sectional view of an embodiment of a breakaway connector apparatus positioned for installation on a needle-free connector.

FIG. 7 illustrates a cross-sectional perspective view of an embodiment of a breakaway connector apparatus installed on a needle-free connector.

FIG. 8 illustrates a cross-sectional perspective view of an embodiment of a breakaway connector apparatus with a breakaway component and needle-free connector detached from the apparatus.

FIG. 9 illustrates a perspective view of an alternative embodiment of a breakaway connector apparatus in accordance with the present disclosure.

FIG. 10 illustrates a cross-sectional perspective view of the embodiment of a breakaway connector apparatus of FIG. 9.

FIG. 11 illustrates a cross-sectional perspective of an alternative embodiment of a breakaway connector apparatus in accordance with the present disclosure.

FIG. 12 illustrates a perspective view of an alternative embodiment of a breakaway connector apparatus in accordance with the present disclosure.

FIG. 13 illustrates a perspective view of an alternative embodiment of a breakaway connector apparatus in accordance with the present disclosure.

FIG. 14 illustrates a perspective view of an embodiment of a breakaway component including a battery, visual indicator and audio indicator.

FIG. 15 illustrates a cross-sectional view of an embodiment of a breakaway connector apparatus including an axially-moveable cannula, a compressible sheath and a battery housing including a battery, and including a pull tab to activate the battery-powered electronic circuit.

FIG. 16 illustrates a cross-sectional view of an embodiment of a breakaway connector apparatus positioned for installation on a needle-free connector.

FIG. 17 illustrates a cross-sectional perspective view of an embodiment of a breakaway connector apparatus installed on a needle-free connector.

FIG. 18 illustrates a cross-sectional view of an embodiment of a breakaway connector apparatus positioned for installation on a needle-free connector.

FIG. 19 illustrates a cross-sectional perspective view of an embodiment of a breakaway connector apparatus installed on a needle-free connector.

FIG. 20 illustrates a cross-sectional view of an embodiment of a breakaway connector apparatus positioned for installation on a needle-free connector.

FIG. 21 illustrates a cross-sectional perspective view of an embodiment of a breakaway connector apparatus installed on a needle-free connector.

FIG. 22 illustrates a cross-sectional perspective view of an embodiment of a breakaway connector apparatus with a breakaway component and needle-free connector detached from the apparatus.

FIG. 23 illustrates a perspective view of an embodiment of a kit for attaching a breakaway connector device to a needle-free connector.

### BRIEF SUMMARY

The present disclosure relates to breakaway connector apparatuses and methods for fluid lines. In some embodiments, the present disclosure provides a method of selecting a breakaway connector apparatus for attachment to a needle-free connector on an intravenous (IV) line attached to a patient. The method includes identifying a push-out force that is associated with the needle-free connector, as well as a desired range of separation force associated with separating a first portion of the IV line from another portion of the IV line. The method further includes selecting a breakaway connector apparatus that includes a housing and a breakaway component that is configured to detach from the housing when a breakaway force is applied to the device, such that a sum of the breakaway force and the push-out force is within the desired range of separation force.

For example, when a breakaway force is applied to the breakaway connector itself, the breakaway component detaches from the housing and a shell of the apparatus. When the apparatus is installed on the needle-free connector, however, the needle-free fitting imparts a push-out force on the apparatus, thereby pushing the housing away from the needle-free fitting and the breakaway component attached thereto. Overall, when a threshold tensile force is applied to the breakaway connector, the needle-free connector and the breakaway component on the device separate together as one unit from the housing and the shell of the apparatus. Upon such an event, a valve in the device blocks incoming fluid flow, and the seal on the needle-free connector blocks outflow from the patient-side tubing assembly.

By taking advantage of identifying the push-out force of the needle-free connector, a breakaway connector apparatus can be selected that includes a proper breakaway force, such that the breakaway force and the push-out force, in sum, result in a threshold tensile force required for separation that is within the desired range of separation force.

In further embodiments, the present disclosure provides a method of using a breakaway connector apparatus. The method includes providing a breakaway connector including a housing and a breakaway component detachably secured to the housing. The breakaway connector includes a breakaway force associated with detaching the housing from the breakaway component. The method further includes providing a needle-free connector including a seal. The method further includes installing the breakaway connector on the needle-free connector. When the breakaway connector is installed on the needle-free connector, the needle-free connector imparts a push-out force on the housing, and a sum of the breakaway force and the push-out force is within a desired range of separation force associated with separating the housing from the needle-free connector.

In further embodiments, the present disclosure provides a breakaway connector apparatus including a breakaway connector. The breakaway connector has a housing, a fixed cannula, and a breakaway component detachably secured to the housing. The apparatus further includes only one valve disposed within the fixed cannula. The breakaway component includes a socket configured for engagement with the needle-free connector, such that the needle-free connector imparts a push-out force on the housing. The breakaway connector includes a breakaway force associated with detaching the breakaway component from the shell. A sum of the breakaway force and the push-out force is within a desired range of separation force associated with separating the shell from the needle-free connector.

Numerous other features and advantages of the present disclosure are set forth in the following description and accompanying figures.

### DETAILED DESCRIPTION

While the making and using of various embodiments of the present invention are discussed in detail below, it should be appreciated that the present invention provides many applicable inventive concepts that are embodied in a wide variety of specific contexts. The specific embodiments discussed herein are merely illustrative of specific ways to make and use the invention and do not delimit the scope of the invention. Those of ordinary skill in the art will recognize numerous equivalents to the specific apparatus and methods described herein. Such equivalents are considered to be within the scope of this invention and are covered by the claims.

In the drawings, not all reference numbers are included in each drawing, for the sake of clarity. In addition, positional terms such as “upper,” “lower,” “side,” “top,” “bottom,” etc. refer to the apparatus when in the orientation shown in the drawing. A person of skill in the art will recognize that the apparatus can assume different orientations when in use.

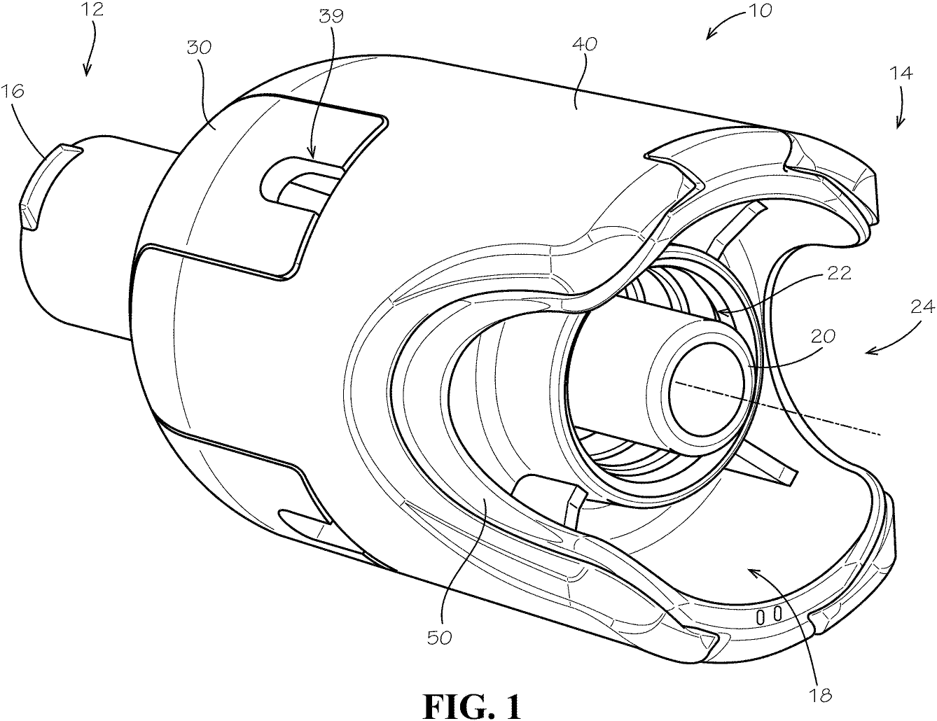

Referring now to FIG. 1, the present disclosure provides a breakaway connector apparatus 10 for attachment to a fluid line. The apparatus 10 includes an input side 12 and an output side 14. The input side 12 may be referred to as a pump side when the device is coupled to an infusion pump. The output side 14 may be referred to as a patient side when the device is coupled to a patient’s IV line. An input fitting 16 includes a male or female luer fitting in some embodiments.

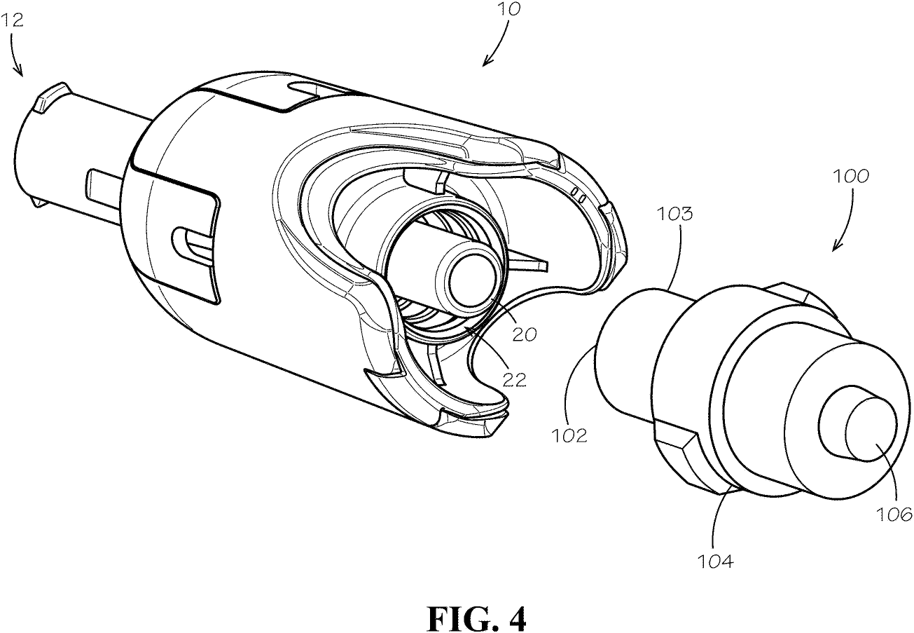

A socket 18 disposed in the apparatus 10 at the output side 14 provides a cavity or recess shaped to accommodate insertion of a needle-free connector on the patient’s tubing set, such as a needle-free connector 100 depicted with reference to FIG. 4. Thus, the apparatus 10 may be installed on (e.g., attached to, engaged with, etc.) the needle-free connector 100. An axially-movable cannula 20 protrudes from the apparatus 10 toward the socket 18 and is positioned to engage a seal on the needle-free connector 100. A first fitting 22 disposed on the output side 14 in the socket 18 includes a female luer fitting in some embodiments. The first fitting 22 is configured to engage a corresponding fitting on the needle-free connector 100 to secure the needle-free connector to the apparatus 10. A device window 24 is defined in the apparatus 10 adjacent to the socket 18. The device window 24 provides access to the socket 18 for manipulating the needle-free connector 100 when received in the socket 18.

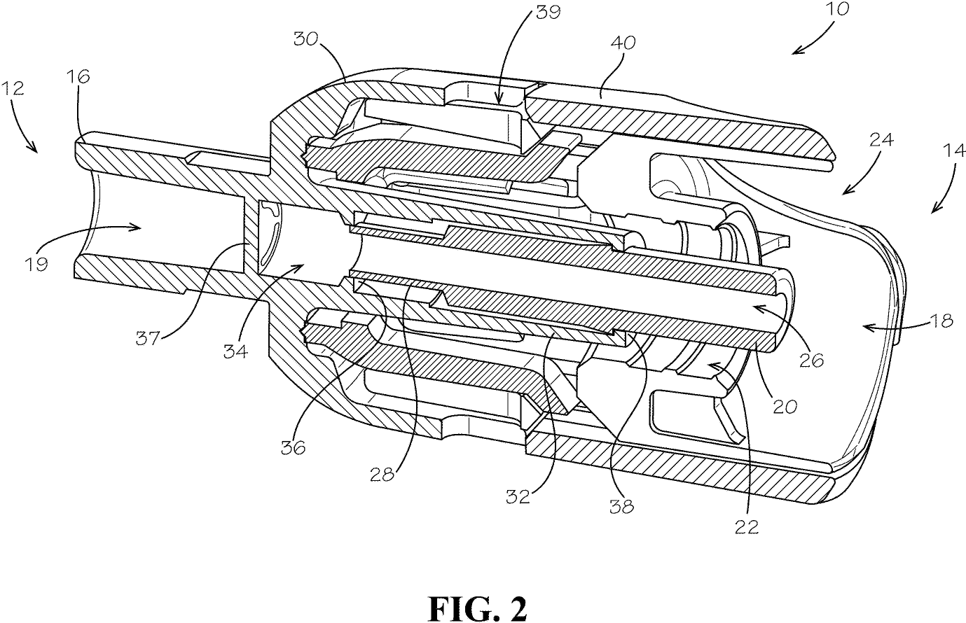

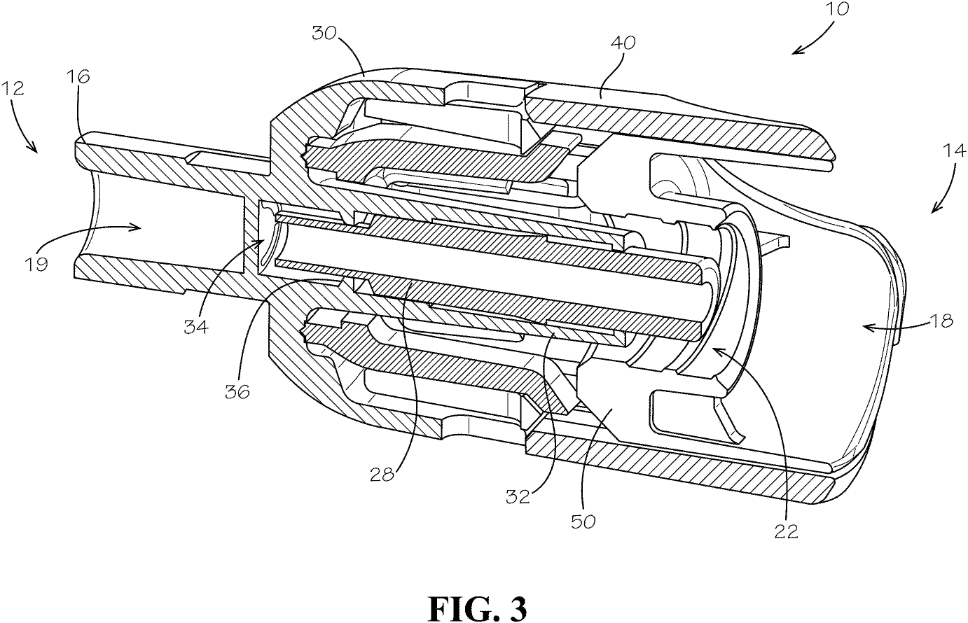

Referring now to FIGS. 2-3, a partial cross-sectional view of the apparatus 10 is shown, according to some embodiments. As shown with reference to FIG. 2, the axially-movable cannula 20 includes an open bore 26 defined axially through the axially-movable cannula 20 from end to end. The bore 26 allows the flow of fluid through the axially-movable cannula 20. The axially-movable cannula 20 has an input end oriented toward the input side 12, and an output end oriented toward the output side 14. The input side of the axially-movable cannula 20 includes a stem 28 having a tapered or reduced radial profile, according to some embodiments.

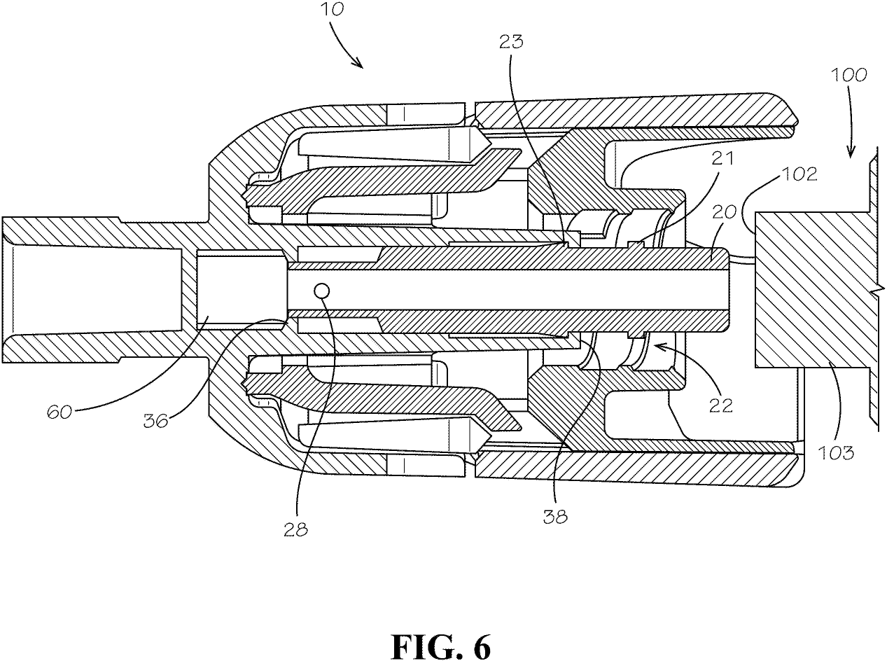

The axially-movable cannula 20 is housed in a fixed cannula 32 on a housing 30. The fixed cannula 32 forms a cylindrical sleeve with an inner channel, and the axially-movable cannula 20 is at least partially positioned inside the inner channel of the fixed cannula 32. The axially-movable cannula 20 is axially moveable inside apparatus 10 by translating back and forth inside the fixed cannula 32 on the housing 30. A valve chamber 34 is defined in the fixed cannula 32 at an input end of the axially-movable cannula 20. An input seal 36 is defined between the stem 28 and the fixed cannula 32 such that the stem 28 may slide axially relative to the fixed cannula 32 without leaking fluid in some embodiments. A valve (such as a valve 60 depicted with reference to FIG. 6) is disposed in the valve chamber 34. The valve 60 is a check valve in some embodiments. The valve 60 can include many forms such as but not limited to an ear plug valve, orchid type valve, duckbill valve, or any other suitable valve known in the art.

During use, the axially-movable cannula 20 may be depressed axially away from the needle-free connector 100 when joined, causing the stem 28 to translate toward and engage the valve contained in the valve chamber 34. The engagement between the stem 28 on the axially-movable cannula 20 and the valve 60 causes the valve 60 to become opened, thereby allowing fluid to travel through inlet 19, into the axially-movable cannula 20 and into the needle-free connector 100.

An example of axial translation of the axially-movable cannula 20 is shown in the relative positioning of the axially-movable cannula 20 in FIGS. 2-3. In some embodiments, a valve chamber wall 37 is disposed in the fixed cannula 32 at an input side of the valve chamber 34. The wall 37 provides a mechanical stop for the valve 60 to prevent the valve 60 from sliding away from the axially-movable cannula 20 when activated. The wall 37 includes openings or perforations to allow fluid flow from the inlet 19 into the bore 26 of the axially-movable cannula 20, according to some embodiments. As described in greater detail below, the axially movable cannula may be separated from the needle-free connector 100 (a “separation event”). When the separation event occurs, the cannula is biased toward the output side and slides back away from the valve chamber to a position as shown in FIG. 2.

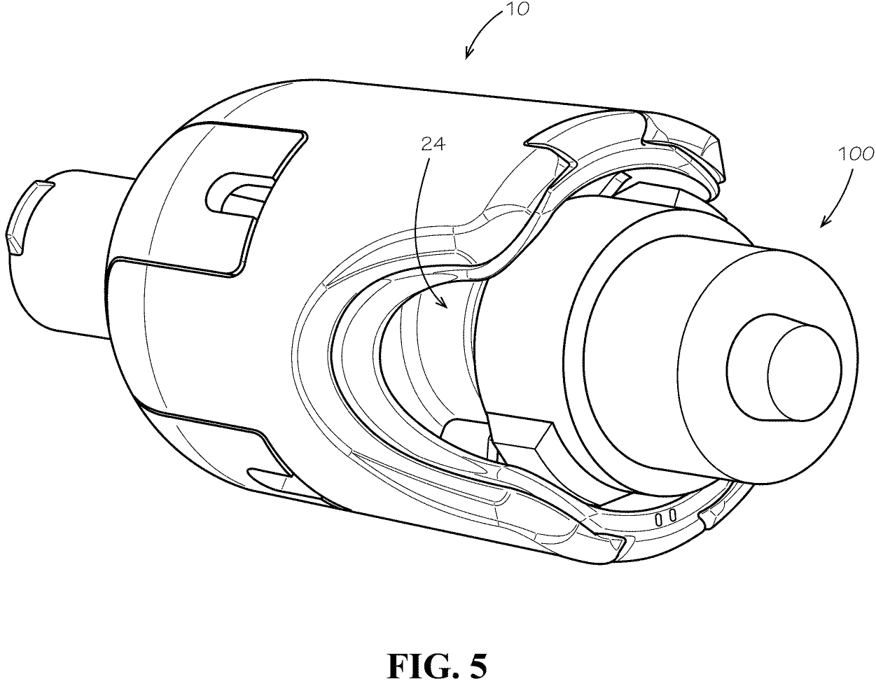

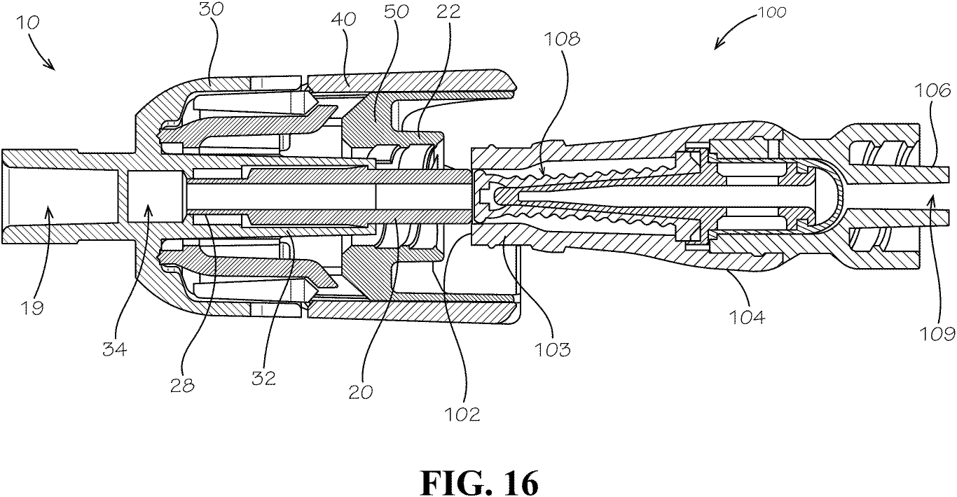

Referring to FIG. 4, the apparatus 10 is shown positioned for installation on the needle-free connector 100, according to some embodiments. The needle-free connector 100 may include any conventional needle-free device, such as a B. Braun Caresite, a B. Braun Ultrasite, a BD Q-Syte, a BD MaxPlus, a BD MaxZero, an ICU Med MicroClave, an ICU Neutron, a Baxter One-Link, a RyMed Invision Plus, or any other suitable needle-free connector known in the art. In some embodiments, the needle-free connector 100 includes an interface 102 including a seal 108 (as shown with reference to FIGS. 16-21, according to varying arrangements). As examples, the seal 108 may include a septum-style seal, an accordion seal, a compressible sheath, a push seal, or any other suitable seal known in the art for a needle-free engagement. The needle-free connector 100 may also include a second fitting 103, such as a male or female luer fitting configured to engage corresponding first fitting 22 on apparatus 10. The needle-free connector 100 may also include a body 104 and a tubing junction 106 extending away from the needle-free connector 100 toward a patient. As described in greater detail below with reference to FIGS. 16-21, the apparatus 10 may accommodate various configurations of the needle-free connector 100, and thus the embodiment of the needle-free connector 100 shown in FIG. 4 (as well as FIGS. 5-8 discussed below) is merely intended as a non-limiting example of the present disclosure.

Referring now to FIGS. 5-7, the needle-free connector 100 is joined directly to breakaway apparatus 10, according to some embodiments. Referring particularly to FIG. 5, the engagement can be viewed through the device window 24. The device window 24 also permits a user to manually twist or turn the needle-free connector 100 if necessary to engage the threaded luer fitting between the components.

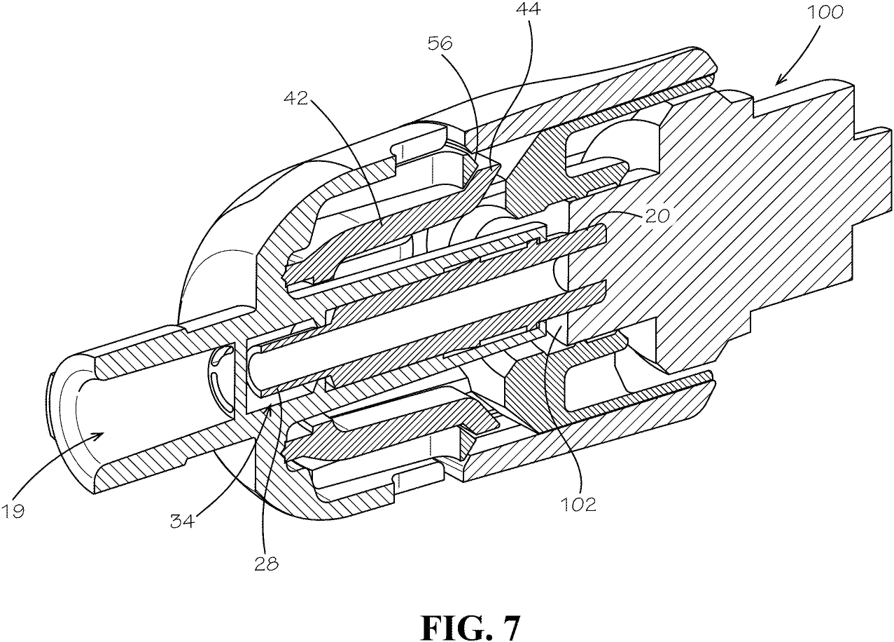

Referring particularly to FIGS. 6-7, prior to installation of the apparatus 10 on the needle-free connector 100, the interface 102 is positioned directly opposite the output end of axially-movable cannula 20. From this position, the second fitting 103 engages with corresponding features on the first fitting 22, thereby causing the axially-movable cannula 20 to penetrate the interface 102, and simultaneously causing the stem 28 to advance into the valve chamber 34, thereby activating and opening the valve 60. The dual action of sliding the axially-movable cannula 20 provides simultaneous or near-simultaneous opening of the seal 108 on the needle-free connector 100 and opening of the valve 60 positioned in valve chamber 34.

Referring further to FIG. 6, in some embodiments, the axially-movable cannula 20 includes a flange 21 protruding radially from the outer surface of the axially-movable cannula 20 outside of fixed cannula 32. The flange 21 provides an axial stop for interface 102 or for other structural features on the needle-free connector 100. When a structure on the needle-free connector 100 engages the flange 21, relative travel between the axially-movable cannula 20 and the needle-free connector 100 stops, and the axially-movable cannula 20 is pushed by needle-free connector 100 toward the valve chamber 34. As the stem 28 passes by the seal 36, the stem 28 engages and opens the valve 60.

In some embodiments, the axially-movable cannula 20 includes a barb 23 protruding from a portion of the axially-movable cannula 20 housed within the fixed cannula 32. The barb 23 provides an axial stop for travel of the axially-movable cannula 20 in a direction away from the valve 60. When the needle-free connector 100 is not engaged with the apparatus 10, the axially-movable cannula 20 is biased away from the valve 60, and the barb 23 engages a channel stop 38 to prevent the axially-movable cannula 20 from sliding too far out of an open end of the inner channel of the fixed cannula 32.

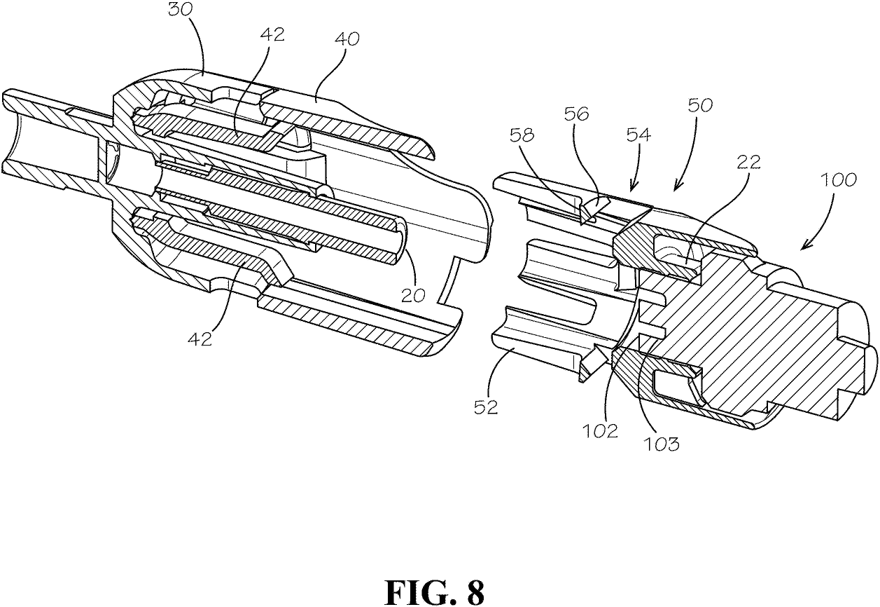

Referring now to FIG. 8, the apparatus 10 is shown detached from the needle-free connector 100, according to some embodiments. When a threshold tensile force associated with the separation event (e.g., a tensile force sufficient to effectuate the separation event) is applied to the IV line, the needle-free connector 100 and a breakaway component 50 may separate together as one unit from a shell 40 and a housing 30. Upon the separation event, the axially-movable cannula 20 will automatically extend away from the valve 60 in the valve chamber 34 by force of the valve 60 pushing the axially-movable cannula 20 or by a coil spring or other biasing member in the fixed cannula 32. Following separation, breakaway component 50 separates fully from shell 40.

In some embodiments, one or more protrusions 52 extend from the breakaway component 50 in a direction away from the needle-free connector 100. The protrusion(s) 52 provide a shield that prevents contamination of the interface 102 on the needle-free connector 100 by mechanically blocking access to the needle-free connector 100. Following the separation event, the breakaway component 50 may be carefully removed from the needle-free connector 100 by unscrewing the threaded luer connection between the breakaway component 50 and the needle-free connector 100.

The threshold tensile force associated with the separation event may be finely tuned by controlling the geometries of the components and the mechanical engagements between breakaway component 50 and shell 40. For example, in some embodiments, the shell 40 includes one or more securing arms 42 extending toward the breakaway component 50. Each of the securing arm(s) 42 include a flexible tip 44 angled radially away from the fixed cannula 32. In further embodiments, the flexible tip 44 may be angled radially toward the fixed cannula 32 in a reversed configuration to achieve the same functionality. Each of the securing arm(s) 42 and the flexible tip(s) 44 may have an independent stiffness defined by material composition, thickness, shape and angle of orientation, among other parameters. Each of the securing arm(s) 42 may deflect toward the fixed cannula 32 as the flexible tip(s) 44 slide past a corresponding ramp 56 on the breakaway component 50 during the separation event, or in a reverse configuration may deflect away from the fixed cannula 32. The inclined angle of the ramp(s) 56 also contribute to a tensile force required to disengage the breakaway component 50 from the apparatus 10, and thus the threshold tensile force associated with the separation event.

In some embodiments, a square wall 58 oriented substantially perpendicular to the longitudinal direction of the apparatus 10 blocks the tips 44 in the event the device is attempted to be re-assembled from the configuration shown with reference to FIG. 8. This feature provides an anti-reconnection function that prevents the breakaway component 50 from being re-inserted into the shell 40. This feature maintains sterility and forces a user to install a new sterile device following the separation event.

In some embodiments, one or more ramp windows 54 (e.g., slots) are aligned with a corresponding one or more openings 39 on the housing 30 (shown with reference to FIG. 1), forming one or more keyholes, to provide access to the securing arms 42. The keyhole access allows a tool to be inserted to depress the securing arms 42, allowing the tips 44 to clear the wall 58 when the breakaway component 50 is initially installed in the shell 40 during manufacture. However, the design prevents a user from being able to deflect the securing arms 42 to attempt to re-connect a used device (e.g., following the separation event).

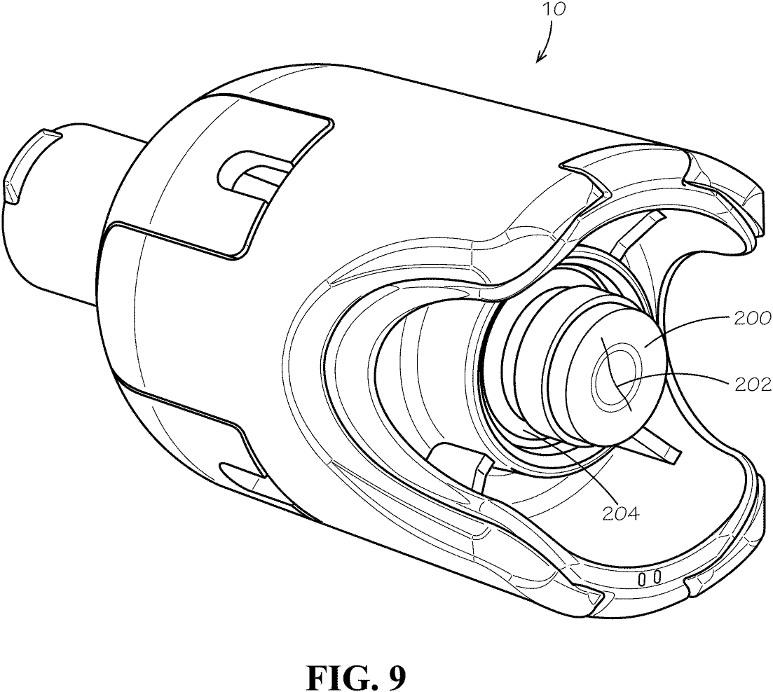

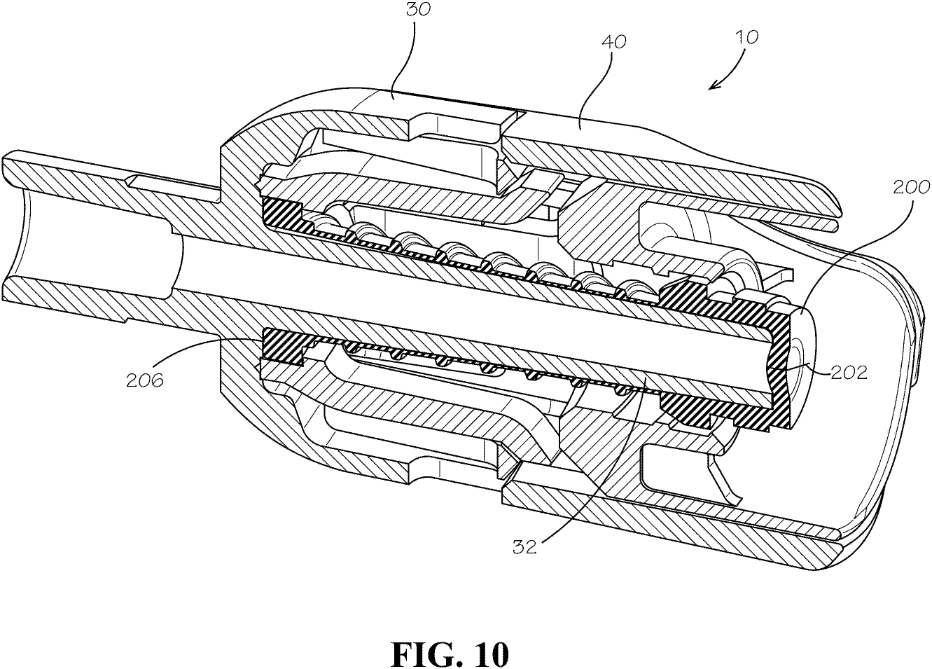

Referring now to FIGS. 9-11, the apparatus 10 includes the fixed cannula 32 with a compressible sheath 200 positioned over the fixed cannula 32, according to some embodiments. The compressible sheath 200 includes an accordion-style compressible sheath formed from a flexible material, such as a plastic, silicone, polymer or elastomer. The compressible sheath 200 includes a seal 202, which may include a split-septum style seal in some embodiments. The seal 202 is biased in a closed position. When the needle-free connector 100 is installed on the apparatus 10, the interface 102 engages the seal 202 and pushes the compressible sheath 200 back over the fixed cannula 32, thereby opening the seal 202 and allowing fluid to travel through the fixed cannula 32 and into the needle-free connector 100. Referring particularly to FIG. 10, when the needle-free connector 100 is installed on the apparatus 10, a distal end of the compressible sheath 200 penetrates the interface 102, thereby deforming and opening the seal 108. In such embodiments, the compressible sheath 200 may be dimensioned and shaped to provide adequate strength and resiliency to engage directly with the interface 102. In other embodiments, the compressible sheath 200 may not be dimensioned and shaped to provide adequate strength and resiliency to engage directly with the interface 102. In such cases, the interface 102 may push the compressible sheath 200 back over the fixed cannula 32, such that, while still opening the seal 202, the compressible sheath 200 does not mechanically engage the seal 108. Accordingly, the fixed cannula 32 (now in direct engagement with the interface 102 due to the compressible sheath 200 being pushed back over the fixed cannula 32) may simultaneously (or near-simultaneously) penetrate the interface 102, thereby deforming and opening the seal 108.

When a separation event occurs, the compressible sheath 200 will spring back to its original shape covering fixed cannula 32 and causing the seal 202 to close, thereby stopping flow out of the fixed cannula 32. The compressible sheath 200 includes the sheath flange 206 extending radially from the base of the compressible sheath 200 in some embodiments. The sheath flange 206 is clamped between the shell 40 and the housing 30, according to some embodiments, in order to secure the axial position of the compressible sheath 200 on the fixed cannula 32. The sheath 200 may take many shapes to achieve the desired functionality.

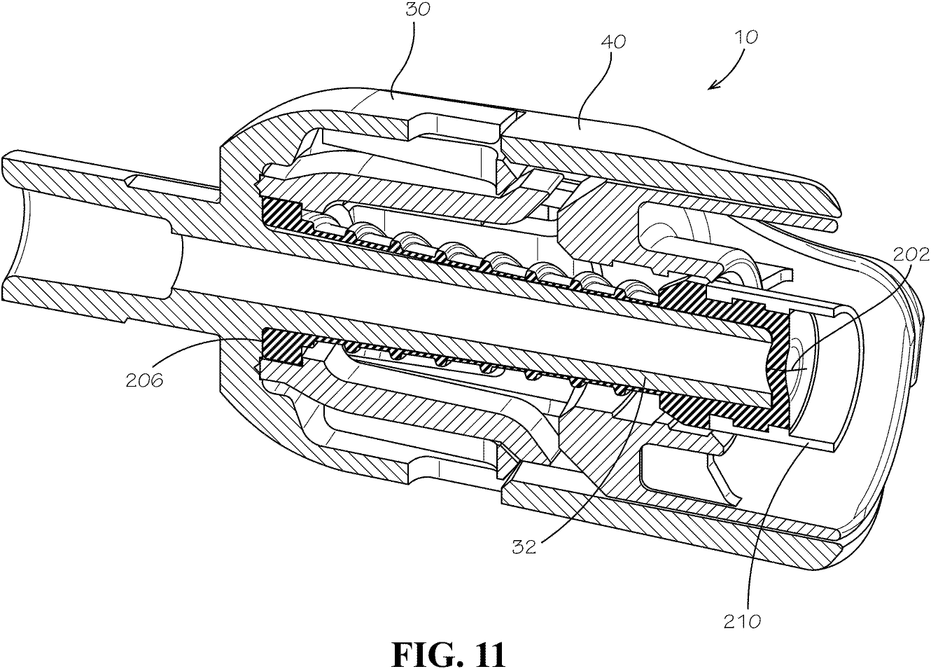

Referring particularly to FIG. 11, a bulkhead 210 is disposed on a distal end of the compressible sheath 200, according to some embodiments. The bulkhead 210 includes an axisymmetric body having an outer wall in the shape of a smooth cylinder, according to some embodiments. In other embodiments, the bulkhead 210 includes other shapes and textures. The bulkhead 210 is formed of a rigid or semi-rigid material, and the bulkhead 210 engages with the needle-free connector 100 when installed on the apparatus 10. The bulkhead 210 functions to translate axial force from the needle-free connector 100 onto the compressible sheath 200 in order to push the compressible sheath 200 backwards over the fixed cannula 32, thereby opening fluid flow through the apparatus 10. In such embodiments, the distal end of the compressible sheath 200 does not contact the interface 102 of the needle-free connector 100 directly, but instead the bulkhead 210 penetrates the interface 102 when the needle-free connector 100 is installed on apparatus 10. The bulkhead 210 is not necessary in all embodiments, and may be omitted in embodiments where the compressible sheath 200 is dimensioned and shaped to provide adequate strength and resiliency to engage directly with the interface 102, or where the needle-free connector 100 is configured such that the fixed cannula 32 engages the interface 102 as mentioned above. In further embodiments, the bulkhead 210 is integrally formed on the compressible sheath 200 as a one-piece construction. Additionally, in some embodiments, the bulkhead 210 is over-molded onto the compressible sheath 200 as a separate component. In other embodiments, the bulkhead 210 is a separate component press-fit onto the tip of the compressible sheath 200. Multiple corresponding radial flanges are provided at the interface between the bulkhead 210 and the compressible sheath 200 in some embodiments. Each flange provides an axial stop to prevent the bulkhead 210 from sliding relative to the outer surface of the compressible sheath 200 when pushed by the needle-free connector 100.

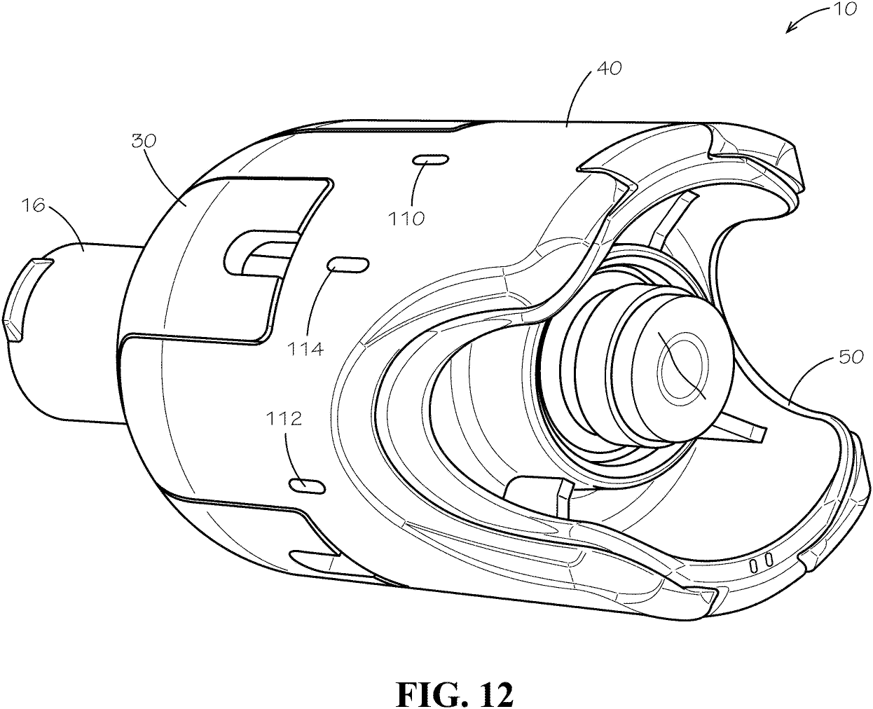

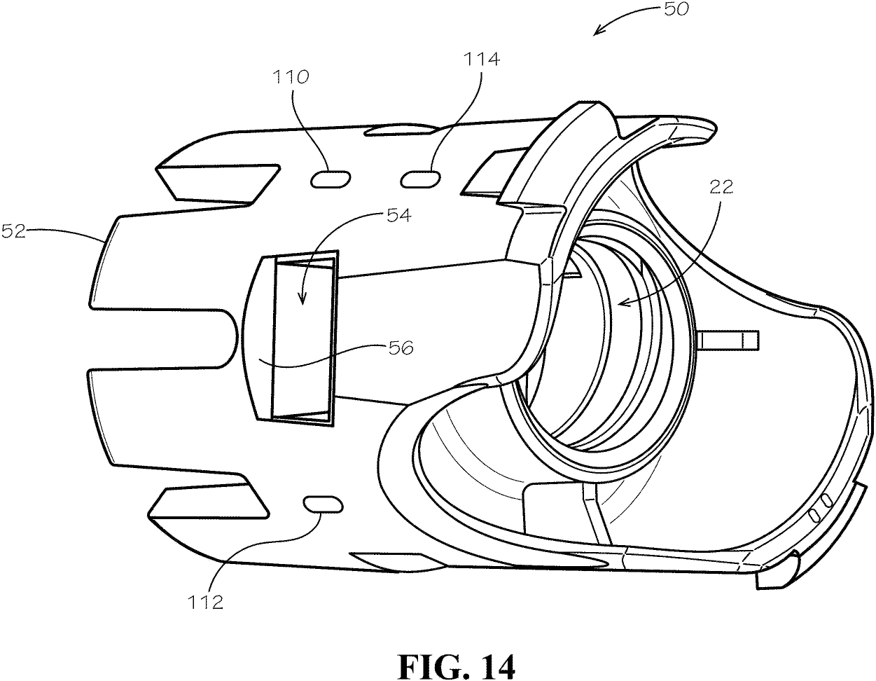

Referring now to FIGS. 12-14, the apparatus 10 is shown to include visual indicia, audio indicator(s), and/or batteries, according to various embodiments. As discussed above, the apparatus 10 may include the housing 30, the shell 40 and the breakaway component 50. The apparatus 10 may be configured to provide one or more visual indications to a user representative of a state condition of the connector. For example, the apparatus 10 may include one or more visual indicia 110 such as a light indicating a status of the apparatus 10. The visual indicia 110 includes an LED light visible to a user in some embodiments. The LED light of the visual indicia 110 may be positioned on the housing 30, the shell 40 or the breakaway component 50 (as shown with particular reference to FIG. 14). In some embodiments, the LED light of the visual indicia 110 is positioned on the surface of the apparatus 10 to provide a visual indication of the status of the apparatus 10 (as shown with particular reference to FIG. 12).

In some embodiments, the LED light of the visual indicia 110 may be configured to display a first color when the device is in a first condition, and a second color when the device is in a second condition. Additionally, the LED light of the visual indicia 110 may provide a blinking pattern to provide a status indicator of the apparatus 10. The LED light of the visual indicia 110 may flash in a first color and pattern. The frequency of the light strobe may increase over time immediately after separation. In some embodiments, the LED light of the visual indicia 110 strobes at one flash every 3 seconds, after 5 minutes it increases to one flash every 2 seconds, after 15 minutes it flashes once every second, etc. After 30 minutes, the LED light of the visual indicia 110 may switch to a pattern of on for 1 second, two flashes/pulses, then back on for one second, and repeat. Various other combinations and patterns may be provided to indicate different state conditions to a user.

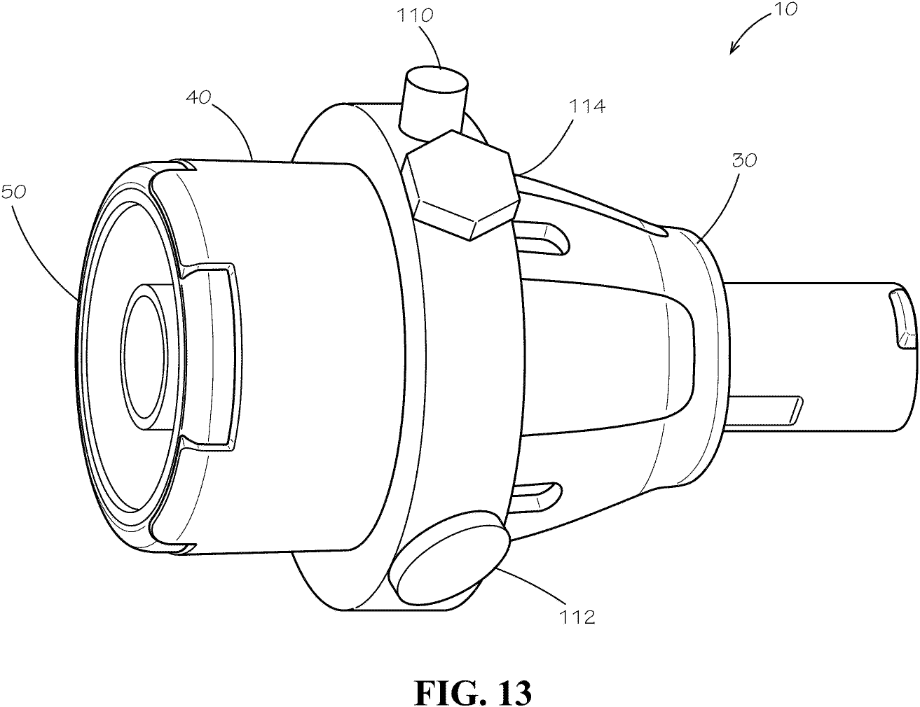

In some embodiments, the visual indicia 110 is mounted inside the apparatus 10 such that the visual indicia 110 is flush with the surface of the connector. In other embodiments, the connector material is translucent, and the visual indicia 110 is embedded within the apparatus 10 such that the light emitted from the LED light of the visual indicia 110 is visible through the material of the apparatus 10. Alternatively, and referring particularly to FIG. 13, the visual indicia 110 may be positioned on an external structure attached to an exterior surface of the apparatus 10. The LED light of the visual indicia 110 may be positioned on an exterior of a ring, and the ring may be installed onto the apparatus 10 as a separate component.

As mentioned above, the apparatus 10 may be configured to include one or more audio indicators. An audio indicator 112 may be configured to provide an auditory signal to a user indicative of a status condition of the apparatus 10. The audio indicator 112 includes a speaker or electronic sound-generating component in some embodiments. The audio indicator 112 is positioned on or near the surface of component 10, in some embodiments. The audio indicator 112 may be configured to emit one or more sounds (e.g., an audio alarm) to indicate a state of the apparatus 10. In some embodiments, following a separate event, the audio alarm provides a first pattern of beeping followed by increasing the rate of the beeping over time. After a predetermined period of time, the audio indicator 112 may switch to different pattern instead of an overly repetitive/metronome like interval (for example beep ...... beep beep ...... beep ...... beep beep beep ..... ).

In some embodiments, apparatus 10 includes both the visual indicia 110 and the audio indicator 112. In other embodiments, the apparatus 10 includes the visual indicia 110 and does not include the audio indicator 112. In further embodiments, the apparatus 10 includes the audio indicator 112 and does not include the visual indicia 110. Various other combinations of one or more visual indicators 110 and audio indicators 112 are provided within the scope of this disclosure.

As mentioned above with reference to FIGS. 12-14, the apparatus 10 may be configured to include one or more batteries. For example, the on-board electronics for the visual indicia 110 and the audio indicator 112 may powered by a battery 114. The battery 114 may be positioned on or near the surface of apparatus 10 (as shown with particular reference to FIG. 12). Alternatively, the battery 114 may be positioned on an external structure such a ring disposed on the apparatus 10 (as shown with particular reference to FIG. 13). In some embodiments, the battery 114 is positioned in a rotational switch, such as a hexagonal component shown in FIG. 13, allowing a user to selectively engage or disengage the battery 114 from the electronics circuit. For example, following a separation event, the hexagonal rotational switch may be rotated to disconnect the battery 114, thereby disabling the visual indicia 110 and/or the audio indicator 112 when the disconnected apparatus 10 is discarded.

Referring particularly to FIG. 14, the visual indicia 110, the audio indicator 112, and/or the batter 114 may be installed on the breakaway component 50, according to some embodiments. As mentioned above with reference to FIG. 1, the apparatus 10 may include a first fitting 22. In some embodiments, the breakaway component 50 includes a first fitting 22, which is configured for attachment to the needle-free connector 100 on the patient’s tubing set. The breakaway component 50 may also include the ramp window(s) 54 and the ramp(s) 56, which are configured to engage corresponding securing arm (s) 42 on the device. The breakaway component 50 may also include the protrusion(s) 52 extending in a direction away from the first fitting 22 to protect the passageway leading to the first fitting 22 and the seal 108 on the needle-free connector 100 when attached. The breakaway component 50, in some embodiments, includes the aforementioned visual indicia 110, which may include an LED light or other electronic component. The visual indicia 110 may be mounted on the body of the breakaway component 50 flush with the exterior surface, according to some embodiments. Alternatively, the visual indicia 110 may be located internal to the breakaway component 50, and light from the LED shines through the material of the body of the breakaway component 50. As a further alternative, the visual indicia 110 may be located on the external surface of the body of the breakaway component 50.

In some embodiments, the breakaway component 50 includes the aforementioned audio indicator 112 configured to emit sound from the device when the breakaway component 50 is separated from the housing 30 and the shell 40. The audio indicator 112 may be mounted flush with the surface of the breakaway component 50, internal to the breakaway component 50, or external to the breakaway component 50, according to various embodiments.

In some embodiments, the breakaway component 50 includes the aforementioned battery 114. When installed on the breakaway component 50, the battery 114 may provide power to the visual indicia 110 and/or the audio indicator 112. The battery 114 may be mounted flush with the surface of the breakaway component 50, internal to the breakaway component 50, or external to the breakaway component 50, according to various embodiments.

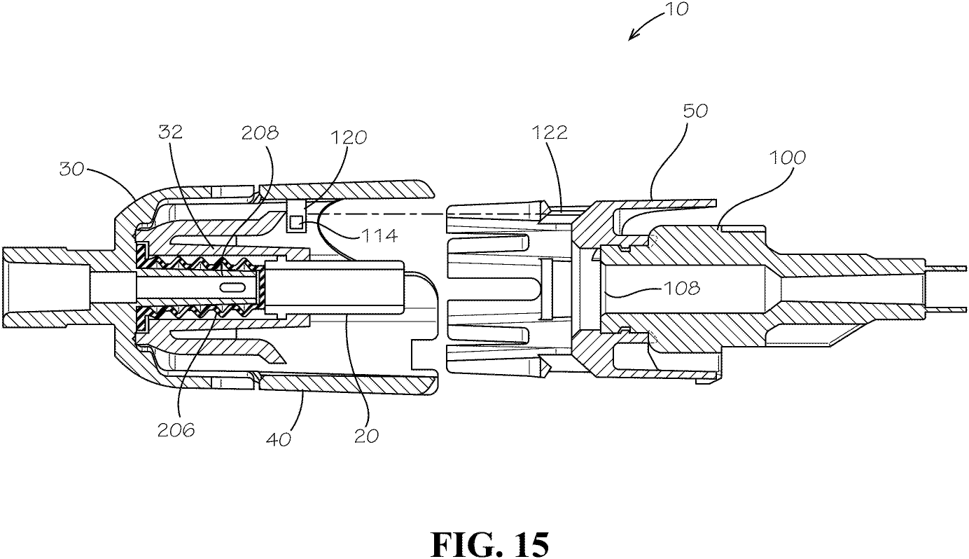

Referring now to FIG. 15, the apparatus 10 is shown to include a compressible sheath, an axially-movable cannula, and/or a pull tab for operating a battery, according to various embodiments. As discussed above, the apparatus 10 includes the housing 30 attached to the shell 40, and the breakaway component 50, which is attached to the needle-free connector 100, such that the apparatus 10 is installed on the needle-free connector 100. The needle-free connector 100 includes the seal 108. In some embodiments, the axially-movable cannula 20 is positioned to translate axially in the fixed cannula 32 adjacent to a valve that is formed by a compressible sheath 206 positioned on a hollow stem 208. When the needle-free connector 100 is secured to the breakaway component 50, the axially-movable cannula 20 compresses the compressible sheath 206, thereby opening the valve formed by the compressible sheath 206 and the hollow stem 208. Simultaneously (or near-simultaneously), the axially-movable cannula 20 opens the seal 108 on the needle-free connector 100. When the threshold tensile force required to actually separate the housing 30 and the shell 40 from the breakaway component 50 and the needle-free connector 100 attached thereto (e.g., effectuating the “separation event”) is applied, the axially-movable cannula 20 translates axially away from the housing 30, thereby closing the valve formed by the compressible sheath 206 and the hollow stem 208. Simultaneously (or near simultaneously), the axially-movable cannula 20 separates from the needle-free connector 100, thereby closing the seal 108.

In some embodiments, a pull tab 122 is fixed to the breakaway component 50 extending toward the housing 30. Prior to the separation event, the pull tab 122 may reside in a battery housing 120 containing the battery 114, thus separating the battery 114 from contacting a battery terminal. Accordingly, prior to the separation event, the battery 114 may be maintained in a zero discharge state disconnected from the electronic circuit for powering components such as visual and/or audio indicators (e.g., the visual indicia 110 and/or the audio indicator 112). For example, as shown with reference to FIG. 12, the visual indicia 110 and/or the audio indicator 112 may be housed on the housing 30 and/or the shell 40. Upon a separation event, the pull tab 122 slides out of the battery housing 120, thereby allowing the battery 114 to engage the electronic circuit onboard the apparatus 10 to provide power to the visual indicia 110 and/or the audio indicator 112. As such, the pull tab 122 operates as a mechanical switch to prevent the battery 114 from contacting its battery terminal and powering the apparatus 10 prior to the separation event, but allowing the battery 114 to contact its battery terminal to power the apparatus 10 following the separation event. As such, the battery 114 may remain in place without discharging prior to the separation event, which may allow for a longer shelf life of the apparatus 10 without discharging the battery 114 before it is needed to power the visual indicia 110 and/or the audio indicator 112.

As mentioned above with reference to FIG. 13, the visual indicia 110 and/or the audio indicator 112 may alternatively be housed on the breakaway component 50. In such embodiments, the pull tab 122 may be positioned on the housing 30 or the shell 40 projecting toward the breakaway component 50. Upon the separation event, the pull tab 122 disengages from a battery housing on the breakaway connector 50. Thus, the shelf-life of the apparatus 10 may be two years or greater due to the pull tab 122 preventing contact between the battery 114 and a corresponding terminal in the electric circuit until a separation event occurs.

In some embodiments, the present disclosure provides a breakaway connector device with only one valve, configured for attachment to a needle-free connector. By providing a device with only one valve, the apparatus may take advantage of the seal on the needle-free connecter to function as a patient-side valve in a separation event.

In further embodiments, the present disclosure provides a method of securing an IV line using the devices disclosed herein.

Referring now to FIGS. 16-21, the apparatus 10 is shown being installed on the needle-free connector 100, according to various embodiments of the present disclosure. As discussed in greater detail below, the apparatus 10 may be configured to accommodate various forces associated with components of the apparatus 10 and the needle-free connector 100. In particular, the apparatus 10 may be configured to accommodate the various forces such that a threshold tensile force required to effectuate the separation event is within a desired range of separation forces.

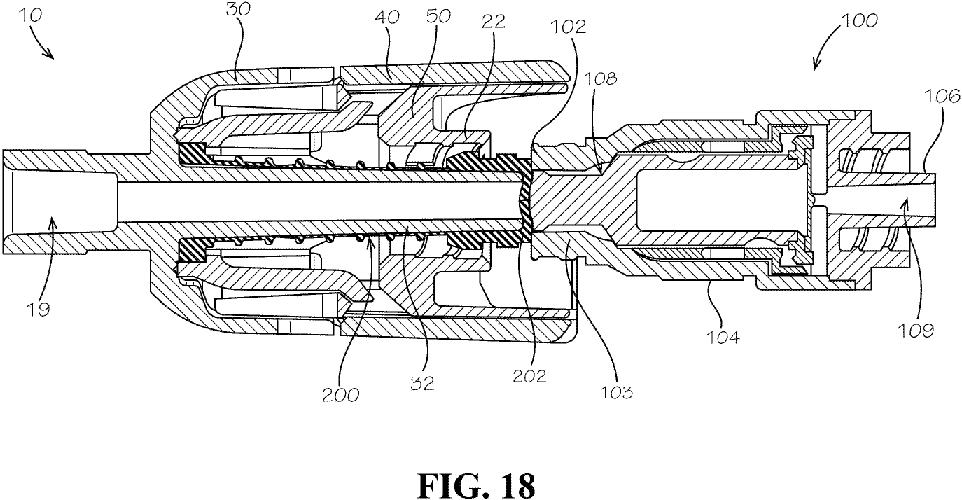

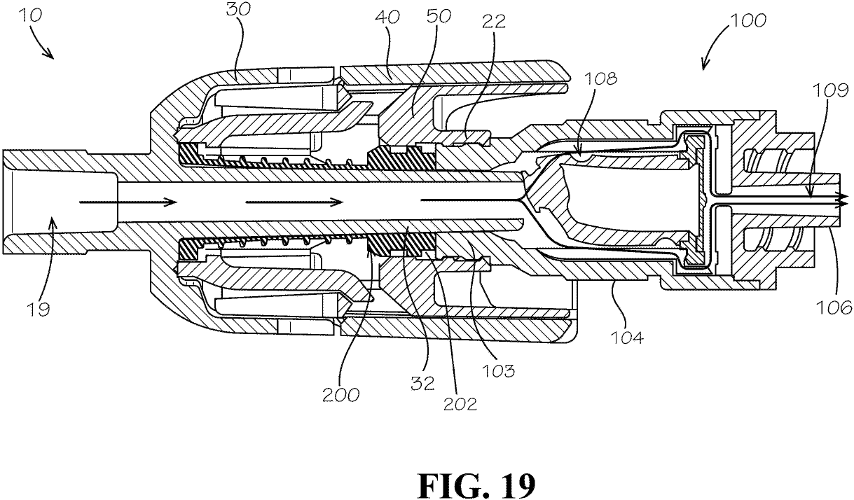

As described above, when the apparatus 10 is installed on the needle-free connector 100, the second fitting 103 of the needle-free connector 100 engages with corresponding features on the first fitting 22 of the apparatus 10, thereby causing a component of the input side 12 (shown with reference to FIG. 1) of the apparatus 10 to open the seal 108 of the needle-free connector 100. As shown according to the various exemplary embodiments depicted herein, the needle-free connector 100 may include the body 104, which houses the seal 108 and forms an outlet 109 within the tubing junction 106. Thus, when the needle-free connector 100 is installed on the apparatus 10 as described herein, the seal 108 may be opened, and a fluid path may be opened from the inlet 19 to the outlet 109. As a first example, as mentioned above with reference to FIGS. 6-7 and 15, and as described in greater detail below with reference to FIGS. 16-17 and 20-21, the axially-movable cannula 20 may open the seal 108. As a second example, as mentioned above with reference to FIGS. 9-11 and described in greater detail below with reference to FIGS. 18-19, the compressible sheath 200 and/or the fixed cannula 32 may open the seal 108. Thus, the component of the input side 12 of the apparatus 10 that opens the seal 108 may be a feature of the housing 30 (e.g., the fixed cannula 32), or a component of the apparatus 10 that is engaged with the housing 30 when the apparatus 10 is installed on the needle-free connector 100 (e.g., the compressible sheath 200, the fixed cannula 32, and/or the axially movable cannula 20).

As described above, various components of the input side 12 may be advanced into the needle-free connector 100, thus penetrating the interface 102 and opening the seal 108, depending on the implementation of the present disclosure. When the seal 108 is opened by a component (“a penetrating component”) of the apparatus 10 as described herein, the seal 108 may correspondingly provide a push-out force on (e.g., push against) the penetrating component. For example, the seal 108 may be biased to a closed configuration (e.g., a configuration where fluid is not allowed to travel through the seal 108 and the outlet 109). As such, the seal 108 may be deformed (elastically, as an example) in order to reach an open configuration (e.g., a configuration where fluid is allowed to travel through the seal 108 and the outlet 109). Accordingly, while the seal 108 is deformed into the open configuration by the penetrating component, the seal 108 may correspondingly provide the push-out force as a result of being biased to return to a pre-deformed state (e.g., the closed configuration), thereby pushing the penetrating component away from the needle-free connector 100 and toward the housing 30. As suggested above, the penetrating component may be a feature of the housing 30, or a component of the apparatus 10 that is engaged with the housing 30 when the apparatus 10 is installed on the needle-free connector 100. Thus, the push-out force may be imparted on the housing 30 by the needle-free connector 100, acting to push the housing 30 away from the needle-free connector 100 while the apparatus 10 is installed on the needle-free connector 100.

As discussed above with reference to FIG. 8, when the threshold tensile force associated with the separation event is applied to the IV line, the needle-free connector 100 and the breakaway component 50 may separate as one unit from the housing 30 and the shell 40. However, a breakaway force that is distinct from the threshold tensile force may be required to separate only the breakaway component 50 from the housing 30 and the shell 40 (effectuating a “detachment event”). For example, the breakaway force associated with the detachment event may be tuned by controlling the geometries and the mechanical engagements between the breakaway component 50 and the shell 40. Of course, the threshold tensile force associated with the separation event may be alternatively understood as the tensile force associated with the detachment event, with the further consideration of the push-out force provided by the needle-free connector 100. Accordingly, in an exemplary scenario where the needle-free connector does not provide a push-out force, the threshold tensile force associated with the separation event would be equivalent to the breakaway force associated with the detachment event. However, as discussed herein, the needle-free connector 100 does in fact provide the push-out force when the apparatus 10 is installed on the needle-free connector 100.

Accordingly, the threshold tensile force associated with the separation event may be a product, at least in part, of the push-out force and the breakaway force associated with the detachment event. For example, the push-out force may act to push the housing 30 away from the needle-free connector 100, which is attached to the breakaway component 50. Accordingly, the push-out force may push on the housing 30 in a manner that is in confluence with the threshold tensile force to be applied in order to effectuate the separation event. In other words, the threshold tensile force required to effectuate the separation event may be the breakaway force reduced by the push-out force. For example, if the breakaway force is about 10 pounds and the push-out force is about 6 pounds, the threshold tensile force required to effectuate the separation event may be about 4 pounds.

In some settings, it would be advantageous to provide the apparatus 10 such that the aforementioned threshold tensile force is within a desired range of separation force associated with the separation event. For example, the desired range of separation force may be a clinically acceptable range of acceptable tensile forces associated with detaching one end of the IV line (such as an end of the IV line attached to the input side 12 of the apparatus 10) from the other end of the IV line (such as an end of the line attached to the needle-free connector 100 and the pump side 14 of the apparatus 10). In this sense, a minimum (or lower end) of the desired range of separation force may be associated with ensuring that minor separation forces do not result in detaching one end of the IV line from the other end of the IV line. Here, cases of typical patient movement may advantageously not result in detaching one end of the IV line from the other end of the IV line. As a corollary, a maximum (or upper end) of the desired range of separation force may be associated with ensuring that substantial separation forces do in fact result in detaching one end of the IV line from the other end of the IV line. Here, cases of substantial (possibly inadvertent) separation forces, such as the patient jerking involuntarily during a procedure, may advantageously result in separating one end of the IV line from the other end of the IV line, thus avoiding harm to the patient that would otherwise occur should the IV line remain intact.

In some embodiments, the desired range of separation force is from about one pound to about six pounds. Such a desired range may be applicable to human patients. In other embodiments, the desired range of separation force is from about ten pounds to fifteen pounds. Such a desired range may be applicable to animal patients that may typically provide greater inadvertent separation forces on the IV line. In other embodiments still, the desired range of separation force is from about one pound to about fifteen pounds (thus corresponding to instances of both human patients and animal patients, as a non-limiting example). It should be appreciated that various desired ranges of separation force may be defined for various patient scenarios, and the examples provided for herein are not intended to limit the present disclosure to any particular range of separation force.

Accordingly, considering the desired range of separation forces discussed herein, the present disclosure provides for a method of using (e.g., a “use method”) a breakaway connector apparatus (such as the apparatus 10). The use method may include a first step of providing a breakaway connector including the housing 30 and the breakaway component 50. As discussed above, the housing 30 may include the fixed cannula 32, and the breakaway connector 50 may include a breakaway force associated with separation of the housing 30 and the shell 40 from the breakaway component 50 (e.g., imparting the “detachment” event). The use method may include a second step of providing the needle-free connector 100. Of course, the needle-free connector 100 may include the seal 108 and a push-out force associated therewith. The use method may include a third step of installing the breakaway connector on the needle-free connector 100, such that a fluid flow path is opened within the fixed cannula 32 and through the seal 108. Thus, via the three use steps provided for herein, the breakaway connector may be installed on the needle-free connector 100, such that the needle-free connector 100 imparts the push-out force on the housing 30, and a sum of the breakaway force and the push-out force (e.g., the breakaway force reduced by the push-out force) is within the desired range of separation force associated with separating the housing 30 from the needle-free connector 100 (e.g., the separation event).

Moreover, the present disclosure thus provides for a breakaway connector apparatus (such as the apparatus 10) for attachment to a needle-free connector (such as the needle-free connector 100). The apparatus 10 may include a breakaway connector that includes the housing 30 (which includes the fixed cannula 32) and the breakaway component 50, which are detachably secured to the housing 30. The apparatus 10 may further include only one valve disposed within the fixed cannula 32. For example, as shown with reference to FIG. 6, the apparatus 10 may include the single valve 60. Additionally, the breakaway component may include the first fitting 22, which is configured for engagement with the second fitting 103 disposed on the needle-free connector 100, such that the needle-free fitting 100 imparts a push-out force on the housing 30. Also, the breakaway connector 50 may include a breakaway force associated with separation of the breakaway component from the shell. Finally, a sum of the breakaway force and the push-out force may be within a desired range of separation force associated with separating the shell 40 from the needle-free fitting 100.

As discussed in greater detail below, the needle-free connector 100 described herein may be embodied by any number of suitable needle-free connectors known in the art. Each of the various needle-free connectors that may embody the needle-free connector 100 may have a distinct push-out force associated therewith. For example, the push-out force associated with a known needle-free connector embodying the needle-free connector 100 may be measured and recorded prior to implementing the needle-free connector in a medical setting. Given the known push-out force of the embodying needle-free connector and the desired range of separation force, an appropriate implementation of the apparatus 10 may be selected in order to ensure that the resulting threshold tensile force that is required to impart the separation event is within the desired range of separation force.

As mentioned above, the needle-free connector 100 described herein may be embodied by any number of suitable needle-free connectors known in the art, each having a distinct push-out force associated therewith. As mentioned above with reference to FIG. 4, the needle-free connector 100 may be embodied by a B. Braun Caresite, a B. Braun Ultrasite, a BD Q-Syte, a BD MaxPlus, a BD MaxZero, an ICU Med MicroClave, an ICU Neutron, a Baxter One-Link, a RyMed Invision Plus, or any other suitable needle-free connector known in the art. In practical application, of course, the various embodying needle-free connectors may provide a range of push-out forces, depending on the particular embodying needle-free connector that is manufactured and provided for implementation on the IV line as described herein. Thus, as explained in greater detail below, the apparatus 10 may be provided (or selected from a number of devices embodying the apparatus 10), such that the breakaway force associated with the apparatus 10 accommodates the entire range of push-out forces associated with the embodying needle-free connector.

Advantageously, the particular push-out force associated with the embodying needle-free connector may not need to be measured prior to implementing the needle-free connector on the IV line. Rather, the various parameters associated with the push-out force (minimum push-out force, maximum push-out force, average push-out force, etc.) provided by the embodying needle-free connector may be known or identified and the apparatus 10 may be provided or selected based on such information. In some embodiments, the apparatus 10 (and the breakaway force associated therewith) is provided or selected based on a known or identified average (or median) push-out force provided by the embodying needle-free connector (among a manufactured sample of particular variety of embodying needle-free connectors, for example). In this sense, given a random embodying needle-free connector of a particular variety, there may be a likelihood that the resulting threshold tensile force associated required to impart the separation event would be safely within the desired range of separation force (towards a median or average of the desired range of separation force, for example).

As a first example, the B. Braun Caresite needle-free connector may, depending on the particular embodying needle-free connector manufactured and implemented, provide a push-out force as high as about 3.4 pounds, as low as about 2.3 pounds, and on average provide a push-out force of about 2.8 pounds. Thus, in order to accommodate attachment to the B. Braun Caresite needle-free connector for a desired range of separation force from about 1 pound to about 6 pounds, it may be advantageous to provide or select the apparatus 10 such that the apparatus 10 provides a breakaway force of about 6.3 pounds. Accordingly, the average B. Braun Caresite needle-free connector, when installed on the apparatus 10, may result in a threshold tensile force of about 3.5 pounds (the 6.3-pound breakaway force reduced by the 2.8-pound average push-out force). In cases where the B. Braun Caresite needle-free connector provides a push-out force as high as about 3.4 pounds, the resulting threshold tensile force may be about 2.9 pounds (the 6.3-pound breakaway force reduced by the 3.4-pound maximum push-out force). In cases where the B. Braun Caresite needle-free connector provides a push-out force as low as about 2.3 pounds, the resulting threshold tensile force may be about 4 pounds (the 6.3-pound breakaway force reduced by the 2.3-pound minimum push-out force). Accordingly, the range of resulting threshold tensile forces associated with separating the housing 30 and the shell 40 from the breakaway component 50 and the needle-free connector 100 attached thereto may be from about 2.9 pounds to about 4 pounds, with an average of 3.5 pounds, thus situating the range of resulting threshold tensile forces required to impart the separation event within the desired range of separation force of from about 1 pound to about 6 pounds.

As a second example, the B. Braun Ultrasite needle-free connector may provide a push-out force as high as about 3.1 pounds, as low as about 1.9 pounds, and on average provide a push-out force of about 2.6 pounds. Thus, in order to accommodate attachment to the B. Braun Ultrasite needle-free connector for a desired range of separation force from about 1 pound to about 6 pounds, the apparatus 10 may be provided or selected such that the apparatus 10 provides a breakaway force of about 6.1 pounds. Accordingly, the average B. Braun Ultrasite needle-free connector, when installed on the apparatus 10, may result in a threshold tensile force of about 3.5 pounds. In cases where the B. Braun Ultrasite needle-free connector provides a push-out force as high as about 3.1 pounds, the resulting threshold tensile force would be about 3 pounds. In cases where the B. Braun Ultrasite needle-free connector provides a push-out force as low as about 1.9 pounds, the resulting threshold tensile force would be about 4.2 pounds. Accordingly, the range of resulting threshold tensile forces required to impart the separation event may be from about 3 pounds to about 4.2 pounds, with an average of 3.5 pounds, thus situating the range of resulting threshold tensile forces required to impart the separation event within the desired range of separation force of from about 1 pound to about 6 pounds.

As a third example, the BD MaxPlus needle-free connector may provide a push-out force as high as about 3.6 pounds, as low as about 2.7 pounds, and on average provide a push-out force of about 3 pounds. Thus, in order to accommodate attachment to the BD MaxPlus needle-free connector for a desired range of separation force from about 1 pound to 6 pounds, the apparatus 10 may be provided or selected to provide a breakaway force of about 6.5 pounds. Accordingly, the BD MaxPlus needle-free connector, when installed on the apparatus 10, may result in a threshold tensile force of about 3.5 pounds on average, as high as about 3.8 pounds, and as low as about 2.9 pounds, situating the range of resulting threshold tensile forces required to impart the separation event within the desired range of separation force from about 1 pound to about 6 pounds.

As a fourth example, the BD Q-Syte needle-free connector may provide a push-out force as high as about 6.7 pounds, as low as about 3.4 pounds, and on average provide a push-out force of about 5.1 pounds. Thus, in order to accommodate attachment to the BD Q-Syte needle-free connector for a desired range of separation force from about 1 pound to 6 pounds, the apparatus 10 may be provided or selected to provide a breakaway force of about 8.6 pounds. Accordingly, the BD Q-Syte needle-free connector, when installed on the apparatus 10, may result in a threshold tensile force of about 2.5 pounds on average, as low as 1.9 pounds, and as high as about 3.4 pounds, situating the range of resulting threshold tensile forces required to impart the separation event within the desired range of separation force from about 1 pound to about 6 pounds.

As a fifth example, the ICU Med Microclave needle-free connector may provide a push-out force as high as about 3.1 pounds, as low about 2.5 pounds, and on average provide a push-out force of about 2.8 pounds. Thus, in order to accommodate attachment to the ICU Med Microclave needle-free connector for a desired range of separation force from about 1 pound to 6 pounds, the apparatus 10 may be provided or selected to provide a breakaway force of about 6.3 pounds. Accordingly, the ICU Med Microclave needle-free connector, when installed on the apparatus 10, may result in a separation force of about 3.5 pounds on average, as low as about 3.2 pounds, and as high as about 3.8 pounds, situating the range of resulting threshold tensile forces required to impart the separation event within the desired range of separation force from about 1 pound to about 6 pounds.

As a sixth example, the ICU Neutron needle-free connector may provide a push-out force as high as about 3.1 pounds, as low as about 2.6 pounds, and on average provide a push-out force of about 2.9 pounds. Thus, in order to accommodate attachment to the ICU Neutron needle-free connector for a desired range of separation force from about 1 pound to 6 pounds, the apparatus 10 may be provided or selected to provide a breakaway force of about 6.4 pounds. Accordingly, the ICU Neutron needle-free connector, when installed on the apparatus 10, may result in a separation force of about 3.5 pounds on average, as low as about 3.3 pounds, and as high as about 3.8 pounds, situating the range of threshold tensile forces required to impart the separation event within the desired range of separation force from about 1 pound to about 6 pounds.

As a seventh example, the Baxter One-Link needle-free connector may provide a push-out force as high as about 2 pounds, as low as about 1 pound, and on average provide a push-out force of about 1.6 pounds. Thus, in order to accommodate attachment to the Baxter One-Link needle-free connector for a desired range of separation force from about 1 pound to 6 pounds, the apparatus 10 may be provided or selected to provide a breakaway force of about 5.1 pounds. Accordingly, the Baxter One-Link needle-free connector, when installed on the apparatus 10, may result in a separation force of about 3.5 pounds on average, as low as about 3.1 pounds, and as high as about 4.1 pounds, situating the range of resulting threshold tensile forces required to impart the separation event within the desired range of separation force from about 1 pound to about 6 pounds.

As an eighth example, the RyMed Invision Plus needle-free connector may provide a push-out force as high as about 2 pounds, as low as about 1 pound, and on average provide a push-out force of about 1.5 pounds. Thus, in order to accommodate attachment to the RyMed Invision Plus needle-free connector for a desired range of separation force from about 1 pound to 6 pounds, the apparatus 10 may be provided or selected to provide a breakaway force of about 5 pounds. Accordingly, the RyMed Invision Plus needle-free connector, when installed on the apparatus 10, may result in a separation force of about 3.5 pounds on average, as low as about 3 pounds, and as high as about 4 pounds, situating the range of resulting threshold tensile forces required to impart the separation event within the desired range of separation force from about 1 pound to about 6 pounds.

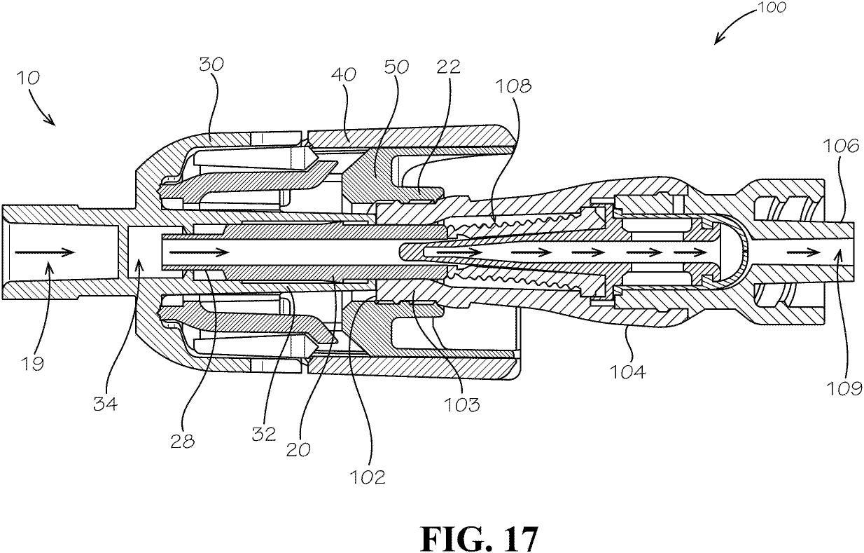

Referring particularly to FIGS. 16-17, the apparatus 10 is shown being installed on the needle-free connector 100, according to some embodiments of the present disclosure. FIGS. 16-17 depict this installation with an embodiment of the apparatus 10 that includes the axially-movable cannula 20 housed in the fixed cannula 32 on the housing 30, as discussed above with reference to FIGS. 6-7. In the exemplary embodiments shown, the needle-free connector 100 includes the seal 108 operating as an accordion seal or a compressible sheath (e.g., an accordion-style compressible sheath) with a split-septum style tip, which is biased to a closed configuration. The seal 108 may be deformed to reach an open configuration by the axially-movable cannula 20 of the apparatus 10 (in other words, the “penetrating component” discussed above is the axially-movable cannula 20). In such embodiments, the seal 108 may be formed from a flexible material, such as a plastic, silicone, polymer, or elastomer.

According to some embodiments, FIG. 16 depicts the apparatus 10 and the needle-free connector 100 prior to installation of the apparatus 10 on the needle-free connector 100, while FIG. 17 depicts the apparatus 10 and the needle-free connector 100 when the apparatus 10 is installed on the needle-free connector 100, thus deforming the seal 108 to reach an open configuration. As shown with particular reference to FIG. 17, the second fitting 103 of the needle-free connector 100 may engage with corresponding features on the first fitting 22, thereby causing the axially-movable cannula 20 to penetrate the interface 102 of the needle-free connector 100. When the axially-movable cannula 20 penetrates the interface 102, the axially-movable cannula 20 may then engage and deform the seal 108, pushing the seal 108 towards the outlet 109, thereby opening the split-septum style tip of the seal 108 and opening a fluid path from the interface 102 to the outlet 109. Simultaneously (or near-simultaneously), the axially-movable cannula 20 may be advanced toward the inlet 19, thus causing the stem 28 to advance into the valve chamber 34, thereby activating and opening the valve 60 within the valve chamber 34 (as discussed above with reference to FIGS. 6-7) and opening a fluid path from the inlet 19 to the axially-movable cannula 20. Thus, a fluid path may be opened from the inlet 19 of the apparatus 10 to the outlet 109 of the needle-free connector 100.

As suggested above, when the seal 108 is deformed to reach an open configuration by the axially-movable cannula 20, the seal 108 may correspondingly provide a push-out force on the axially-movable cannula 20 as a result of being biased to return to a closed configuration, thereby pushing the axially-movable cannula 20 away from the needle-free connector 100 and toward the housing 30. In some embodiments, the push-out force is transferred by the axially-movable cannula 20 to the housing 30, such that, overall, the seal 108 imparts the push-out force on the housing 30. As an example, when the stem 28 activates and opens the valve 60, the valve 60 may be biased against the valve chamber 34 of the housing 30 towards the inlet 19. As another example, when the axially-movable cannula 20 is advanced toward the inlet 19, an outer surface of the axially-movable cannula 20 may provide a frictional force on an inner surface of the fixed cannula 32 of the housing 30 in the direction of the inlet 19. As another example still, in order for the stem 28 to activate and open the valve 60, the stem 28 may push against, or provide a frictional force against, the input seal 36 of the housing 30 towards the inlet 19. Thus, the seal 108 may impart the overall push-out force on the housing 30. Accordingly, in some embodiments, and as discussed above, the threshold tensile force required to effectuate the separation event may be the breakaway force (which may be tuned by controlling the geometries and the mechanical engagements between the breakaway component 50 and the shell 40 of the apparatus 10, as mentioned above with reference to FIG. 8), reduced by the push-out force.

In other embodiments, however, the threshold tensile force required to effectuate the separation event may be the breakaway force reduced by the push-out force, additionally increased by a biasing force imparted by the apparatus 10. For example, when the stem 28 activates and opens the valve 60, the valve 60 may provide a biasing force against the stem 28, therefore pushing the axially-movable cannula 20 toward the needle-free connector 100. Thus, where the valve 60 provides such a biasing force against the stem 28, the threshold tensile force required to effectuate the separation event may then be the breakaway force reduced by the push-out force, additionally increased by the biasing force provided by the valve 60 of the apparatus 10.

As mentioned above, given the known push-out force of the needle-free connector 100 as depicted with reference to FIGS. 16-17, and a desired range of separation force, an appropriate implementation of the apparatus 10 may be provided in order to ensure that the resulting threshold tensile force that is required to impart the separation event is within the desired range of separation force.

Referring particularly to FIGS. 18-19, the apparatus 10 is shown being installed on the needle-free connector 100, according to some embodiments of the present disclosure. FIGS. 18-19 depict this installation with an embodiment of the apparatus 10 that includes the compressible sheath 200 positioned over the fixed cannula 32, as discussed above with reference to FIGS. 9-11. In the exemplary embodiments shown, the needle-free connector 100 includes the seal 108 operating as a push seal, which is biased to a closed configuration. The seal 108 may be deformed to reach an open configuration by the compressible which is biased to a closed position and is opened by the compressible sheath 200 of the apparatus 10. In such embodiments, the seal 108 may be formed from a flexible material, such as a plastic, silicone, polymer, or elastomer.

According to some embodiments, FIG. 18 depicts the apparatus 10 and the needle-free connector 100 prior to installation of the apparatus 10 on the needle-free connector 100, while FIG. 19 depicts the apparatus 10 and the needle-free connector 100 when the apparatus 10 is installed on the needle-free connector 100, thus opening the seal 108 of the needle-free connector 100. As shown with particular reference to FIG. 19, the second fitting 103 of the needle-free connector 100 engages with corresponding features on the first fitting 22, thereby causing the compressible sheath 200 to engage the interface 102.

In some embodiments, as discussed in greater detail above with reference to FIGS. 9-11 and as shown with reference to FIG. 19, the compressible sheath 200 may not be dimensioned and shaped to provide adequate strength and resiliency to penetrate the interface 102. In such embodiments, when the compressible sheath 200 engages the interface 102, the interface 102 may push the compressible sheath 200 back over the fixed cannula 32, thereby opening the seal 202 and allowing fluid to travel from the inlet 19 through the fixed cannula 32. Simultaneously (or near-simultaneously), the fixed cannula 32 may penetrate the interface 102 in order to engage and deform the seal 108, thereby allowing fluid to travel from the interface 102 to the outlet 109 (in other words, the “penetrating component” discussed above is the fixed cannula 32). Thus, a fluid path may be opened from the inlet 19 to the outlet 109.

In other embodiments, as discussed in greater detail above with reference to FIGS. 9-11, the compressible sheath 200 may be dimensioned and shaped to provide adequate strength and resiliency to engage directly with the interface 102. In such embodiments, when the needle-free connector 100 is installed on the apparatus 10, a distal end of the compressible sheath 200 may engage the interface 102, thereby opening the seal 202 and allowing fluid to travel from the inlet 19 through the compressible sheath 200. Simultaneously (or near-simultaneously), the distal end of the compressible sheath 200 may penetrate the interface 102 in order to engage end deform the seal 108, thereby allowing fluid to travel from the interface 102 to the outlet 109 (in other words, the “penetrating component” discussed above is the compressible sheath 200). Thus, a fluid path may be opened from the inlet 19 to the outlet 109 of the needle-free connector 100.

As suggested above, when the seal 108 is deformed to reach an open configuration opened by the compressible sheath 200 and/or the fixed cannula 32, the seal 108 may correspondingly provide a push-out force on the compressible sheath 200 and/or the fixed cannula 32 as a result of being biased to return to a closed configuration.

In some embodiments where the compressible sheath 200 is pushed back over the fixed cannula 32 such that the compressible sheath 200 does not penetrate the interface 102 (as shown with reference to FIG. 19), the push-out force may be imparted (in portions, for example) on both the compressible sheath 200 (which transfers the push-out force to the housing 30) and the fixed cannula 32 of the housing 30 (as well as the bulkhead 210, in some cases). As a first example, when the compressible sheath 200 is pushed back over the fixed cannula 32, the compressible sheath 200 may become compressed, thus transferring a first portion of the push-out force to the housing 30 at or near the base of the fixed cannula 32 (e.g., where the compressible sheath 200 forms the sheath flange 206). As a second example, the seal 108 may impart a second portion of the push-out force directly on the fixed cannula 32 of the housing 30 due to the mechanical engagement between the fixed cannula 32 and the seal 108 that opens the seal 108. Thus, the seal 108 may impart the overall push-out force on the housing 30.

In other embodiments where the compressible sheath 200 does penetrate the interface 102, the push-out force may be imparted on the compressible sheath 200, which transfers the push-out force to the housing 30. For example, the compressible sheath 200 may become compressed, thus transferring the push-out force to the housing 30 at or near the base of the fixed cannula 32 (e.g., where the compressible sheath 200 forms the sheath flange 206). Thus, the seal 108 may impart the overall push-out force on the housing 30.

Accordingly, in some embodiments, and as discussed above, the threshold tensile force required to effectuate the separation event may be the breakaway force (which may be tuned by controlling the geometries and the mechanical engagements between the breakaway component 50 and the shell 40 of the apparatus 10, as mentioned above with reference to FIG. 8), reduced by the push-out force.

In other embodiments, however, the threshold tensile force required to effectuate the separation event may be the breakaway force reduced by the push-out force, additionally increased by a biasing force imparted by the apparatus 10. For example, when the compressible sheath 200 is pushed back over the fixed cannula 32, the compressible sheath 200 may become compressed, thus providing a biasing force against the interface 102. Thus, where the compressible sheath 200 provides such a biasing force against the interface 102, the threshold tensile force required to effectuate the separation event may then be the breakaway force reduced by the push-out force, additionally increased by the biasing force provided by the compressible sheath 200 of the apparatus 10.

As mentioned above, given the known push-out force of the needle-free connector 100 as depicted with reference to FIGS. 18-19, and a desired range of separation force, an appropriate implementation of the apparatus 10 may be provided in order to ensure that the resulting threshold tensile force that is required to impart the separation event is within the desired range of separation force.

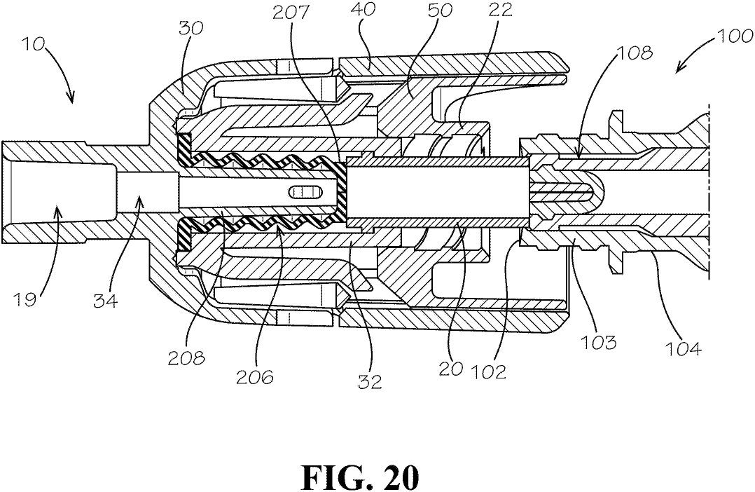

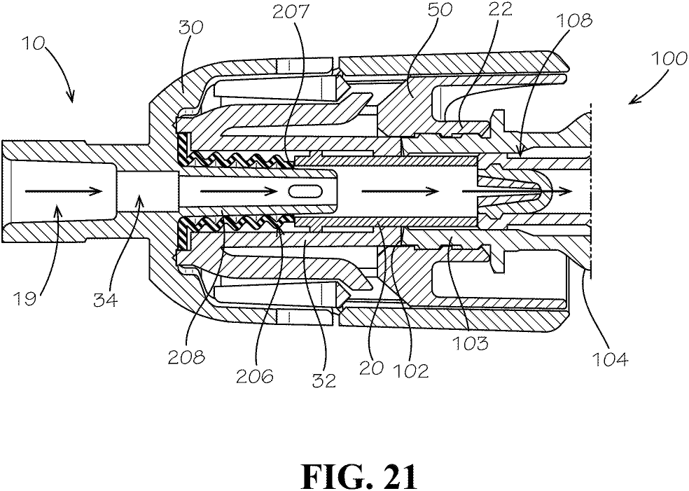

Referring particularly to FIGS. 20-21, the apparatus 10 is shown being installed on the needle-free connector 100, according to some embodiments of the present disclosure. FIGS. 20-21 depict this installation with an embodiment of the apparatus 10 that includes the axially-movable cannula 20 positioned to translate axially in the fixed cannula 32 adjacent to a valve that is formed by the compressible sheath 206 positioned on the hollow stem 208, as discussed above with reference to FIG. 15. In the exemplary embodiments shown, the needle-free connector 100 includes the seal 108 with a v-shaped seam. The v-shaped seam may be biased to an open position, while the surrounding material of the seal 108 compresses the v-shaped seam to a closed position and is biased to retain the v-shaped seam near the interface 102, as shown with particular reference to FIG. 20. The seal 108 may be deformed to reach an open configuration by the axially-movable cannula 20 of the apparatus 10 (in other words, the “penetrating component” discussed above is the axially-movable cannula 20). In such embodiments, the v-shaped seam within the seal 108 may be formed of a rigid material such as nylon or plastic, while the remaining portions of the seal 108 may be formed from a flexible material, such as a plastic, silicone, polymer, or elastomer.

According to some embodiments, FIG. 20 depicts the apparatus 10 and the needle-free connector 100 prior to installation on the apparatus 10 to the needle-free connector 100, while FIG. 21 depicts the apparatus 10 and the needle-free connector 100 when the apparatus 10 is installed on the needle-free connector 100, thus deforming the seal 108 to reach an open configuration. As shown with particular reference to FIG. 21, the second fitting 103 of the needle-free connector 100 may engage with corresponding features on the first fitting 22, thereby causing the axially-movable cannula 20 to penetrate the interface 102 of the needle-free connector 100. When the axially-movable cannula 20 penetrates the interface 102, the axially-movable cannula 20 may then engage the v-shaped seam within the seal 108, deforming the surrounding material of the seal 108 and pushing the v-shaped seam away from the interface 102 and towards the outlet 109, such that the v-shaped seam is allowed to open therefore allowing the v-shaped seam to move the seal 108 into an open configuration. Accordingly, a fluid path may be opened from the interface 102 to the outlet 109. Simultaneously (or near-simultaneously), the axially-movable cannula 20 compresses the compressible sheath 206, thereby opening the valve formed by the compressible sheath 206 and the hollow stem 208 and opening a fluid path from the inlet 19 to the axially movable cannula 20 (as discussed above with reference to FIG. 15). Thus, a fluid path may be opened from the inlet 19 of the apparatus 10 to the outlet 109 of the needle-free connector 100.

As suggested above, when the seal 108 is deformed to reach an open configuration by the axially-movable cannula 20, the surrounding material of the seal 108 is biased to move the v-shaped seam away from the outlet 109 such that the v-shaped seam is retained near the interface 102. Thus, the seal 108 may correspondingly provide a push-out force on the axially-movable cannula 20 as a result of being biased to return to a closed configuration, thereby pushing the axially-movable cannula 20 away from the needle-free connector 100 and toward the housing 30. In some embodiments, the push-out force is transferred by the axially-movable cannula 20 to the housing 30, such that, overall, the seal 108 imparts the push-out force on the housing 30. As an example, when the axially-movable cannula 20 compresses the compressible sheath 206, the compressible sheath 206 may, in turn, push against the housing 30 in the direction of the inlet 19. As another example, when the axially-movable cannula 20 is advanced toward the inlet 19, an outer surface of the axially-movable cannula 20 may provide a frictional force on an inner surface of the fixed cannula 32 of the housing 30 in the direction of the inlet 19. Thus, the seal 108 may impart the overall push-out force on the housing 30. Accordingly, in some embodiments, and as discussed above, the threshold tensile force required to effectuate the separation event may be the breakaway force (which may be tuned by controlling the geometries and the mechanical engagements between the breakaway component 50 and the shell 40 of the apparatus 10, as mentioned above with reference to FIG. 8), reduced by the push-out force.

In some embodiments, however, the threshold tensile force required to effectuate the separation event may be the breakaway force reduced by the push-out force, additionally increased by a biasing force imparted by the apparatus 10. For example, when the axially-movable cannula 20 compresses the compressible sheath 206, the compressible sheath 206 may provide a biasing force against the axially-movable cannula 20, therefore pushing the axially-movable cannula 20 toward the needle-free connector 100. Thus, where the compressible sheath 206 provides such a biasing force against the axially-movable cannula 20, the threshold tensile force required to effectuate the separation event may then be the breakaway force reduced by the push-out force, additionally increased by the biasing force provided by the compressible sheath 206 of the apparatus 10.

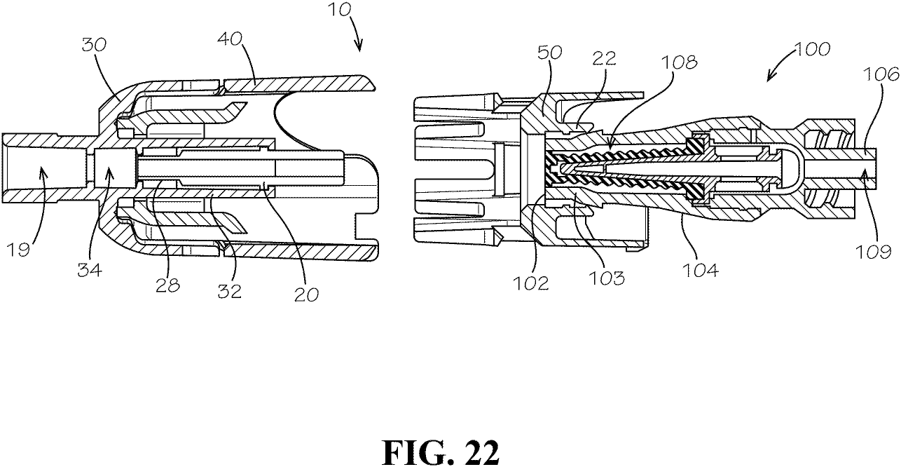

Referring now to FIG. 22, the apparatus 10 is shown detached from the needle-free connector 100, according to some embodiments. In the exemplary embodiments shown, the needle-free connector 100 includes the seal 108 operating as an accordion seal or a compressible sheath (e.g., an accordion-style compressible sheath) with a split-septum style tip as discussed above with reference to FIGS. 16-17. When a threshold tensile force associated with the separation event is applied to the IV line, the needle-free connector 100 and a breakaway component 50 may separate together as one unit from the shell 40 and the housing 30. Upon the separation event, the axially-movable cannula 20 may automatically extend away from the valve 60 in the valve chamber 34 by force of the valve 60 pushing the axially-movable cannula 20 or by a coil spring or other biasing member in the fixed cannula 32. Following separation, the breakaway component 50 separates fully from the shell 40.

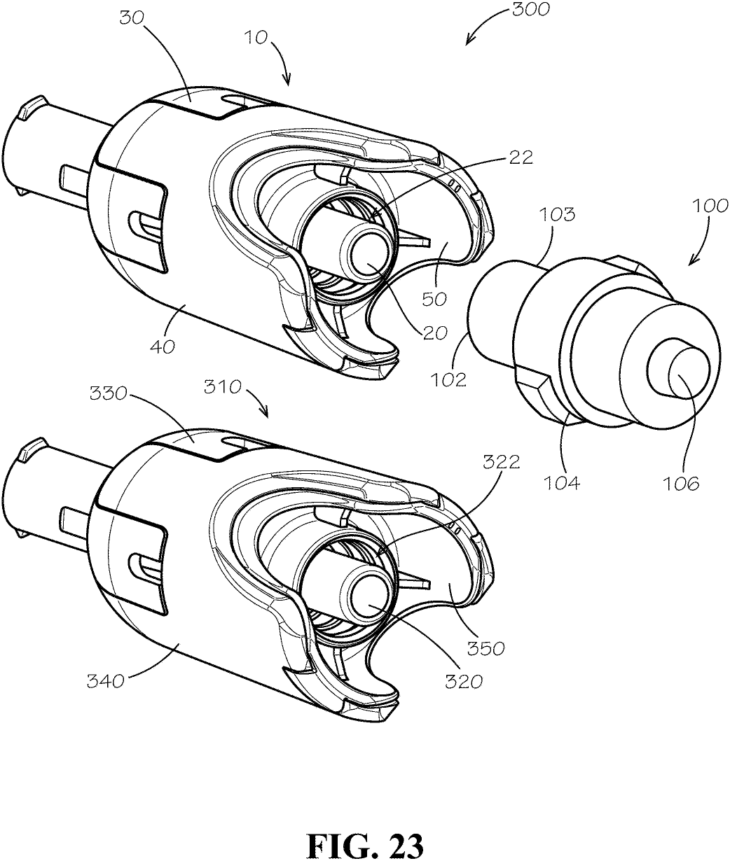

Referring now to FIG. 23, a kit 300 of breakaway connector apparatuses is shown, according to some embodiments. As mentioned above, given a known push-out force (or statistical parameters associated therewith) associated with a needle-free connector, one of multiple breakaway connector apparatuses provided in the kit 300 may be selected in order to ensure that, when the selected breakaway connector apparatus is attached to the needle-free connector, the resulting threshold tensile force sufficient to effectuate the separation event is within the desired range of separation force.

In some embodiments, the kit 300 includes two or more breakaway connector apparatuses as described herein. However, the breakaway connector apparatus may provide varying breakaway forces associated therewith. For example, the kit 300 may include the apparatus 10, as well as a breakaway connector apparatus (apparatus) 310, which, in a corresponding fashion apparatus 10, includes a housing 330, a shell 340, a fitting 322, a breakaway component 350, and an axially-movable cannula 320.

As mentioned above, the breakaway force associated with an embodiment of the apparatus 10 may be tuned by controlling the geometries and the mechanical engagements between the breakaway component 50 and the shell 40. Similarly, the breakaway force associated with the apparatus 310 may be tuned by controlling the geometries and the mechanical engagements between the breakaway component 350 and the housing 340. Thus, multiple implementations of the apparatus 10 may be provided with varying breakaway forces included therewith that are known to a user of the kit 300. It should be appreciated that, while the kit 300 as depicted includes two breakaway connector apparatuses, the kit 300 can include any number of breakaway connector apparatuses in order to provide a wide range of associated breakaway forces that can be applied to various needle-free fittings in order to achieve threshold tensile forces that satisfy varying desired ranges of separation force associated with the separation event.

Accordingly, given the multiple implementations of the apparatus 10 that may be provided with various breakaway forces associated therewith, the present disclosure provides for a method of selecting a breakaway connector device (such as the apparatus 10 and the apparatus 310) for attachment to a needle-free connector (such as the needle-free connector 100) on an IV line (e.g., a “selection method”). The selection method may include a first step of providing the needle-free connector 100 for attachment to a breakaway connector. For example, the needle-free connector 100 may be acquired, or already installed on the IV line at the inception of the selection method. Of course, the needle-free connector 100 may have a push-out force associated therewith. The selection method may include a second step of identifying the push-out force imparted by the needle-free connector 100. For example, the push-out force may be identified by measuring the push-out force directly, or the push-out force (or statistical parameters associated therewith) may simply be known (e.g., on record as associated with the particular embodiment of the needle-free connector 100). In this sense, the push-out force may be measured or known as a value such as about 2 pounds, as an example.

The selection method may include a third step of identifying a desired range of separation force associated with separating a first portion of the IV line (such as the input side 12) from a second portion of the IV line (such as the output side 14). For example, the desired range of separation force may be from about 1 pound to about 6 pounds. The selection method may include a fourth step of selecting a breakaway connector device (e.g., the apparatus 10, the apparatus 310, etc.), such that a sum of the breakaway force and the push-out force (e.g., the breakaway force reduced by the push-out force) is within the desired range of separation force. As a non-limiting example, the apparatus 10 may include a breakaway force of about 5 pounds, and the apparatus 310 may include a breakaway force of about 10 pounds. Thus, attaching the apparatus 10 would result in a threshold tensile force sufficient for a separation event of about 3 pounds, while the apparatus 310 would result in a corresponding threshold tensile force of about 8 pounds, in this non-limiting example. Accordingly, the apparatus 10 may be selected, since the resulting threshold tensile force sufficient for a separation event of about 3 pounds is within the desired range of separation force, while the corresponding threshold tensile force of about 8 pounds is not.

Moreover, the present disclosure thus provides for a kit, such as the kit 300, for attaching a breakaway connector device to a needle-free connector (such as the needle-free connector 100) that imparts a push-out force. The kit 300 may include a first breakaway connector, such as the apparatus 10, having a first housing (the housing 30) and a first breakaway component (the breakaway component 50). The apparatus 10 thus provides a first breakaway force associated with separation of the first housing from the first breakaway component. The kit 300 may further include a second breakaway connector, such as the apparatus 310, having a second housing (the housing 330) and a second breakaway component (the breakaway component 350). The apparatus 310 thus provides a second breakaway force associated with separation of the second housing from the second breakaway component. Due to the aforementioned tuning that may differentiate breakaway forces between various breakaway connector apparatuses, the second breakaway force may be different than the first breakaway force. Each of the apparatuses 10, 310 are configured for attachment to the needle-free connector 310, and at least one of the first breakaway force and the second breakaway force, when combined with the push-out force imparted by the needle-free fitting 100, provides a sum that is within a desired range of separation force associated with separating the first housing or the second housing, respectively, from the needle-free fitting 100.

Thus, although there have been described particular embodiments of the present invention of new and useful devices and methods, it is not intended that such references be construed as limitations upon the scope of this invention except as set forth in the claims.

What is claimed is:

1. A method of selecting a breakaway connector apparatus for attachment to a needle-free connector on an intravenous (IV) line, comprising:
providing a needle-free connector for attachment to a breakaway connector, wherein the needle-free connector includes a push-out force associated with the needle-free connector;
identifying the push-out force imparted by the needle-free connector;
identifying a desired range of separation force associated with separating a first portion of the IV line from a second portion of the IV line; and
selecting a breakaway connector apparatus including a housing and a breakaway component that is configured to detach from the housing when a breakaway force is applied to the device, such that a sum of the breakaway force and the push-out force is within the desired range of separation force.

2. The method of claim 1, wherein the desired range of separation force is from approximately one pound to fifteen pounds.

3. The method of claim 1, wherein the desired range of separation force is from approximately one pound to six pounds.

4. A method of using a breakaway connector apparatus, comprising:
providing a breakaway connector including a housing and a breakaway component detachably secured to the housing, the housing having a fixed cannula, wherein the breakaway connector includes a breakaway force associated with detaching the housing from the breakaway component;
providing a needle-free connector including a seal; and
installing the breakaway connector on the needle-free connector, such that a fluid path is opened within the fixed cannula and through the seal in the needle-free connector,
wherein when the breakaway connector is installed on the connector, the needle-free connector imparts a push-out force on the housing, and a sum of the breakaway force and the push-out force is within a desired range of separation force associated with separating the housing from the needle-free connector.

5. The method of claim 4, wherein the breakaway connector further includes an axially-movable cannula disposed within the fixed cannula,
wherein when the breakaway connector is installed on the needle-free connector, the axially-movable cannula opens the seal in the needle-free connector.

6. The method of claim 5, wherein the breakaway connector further includes a valve disposed within the fixed cannula, and
wherein when the breakaway connector is installed on the needle-free connector, the axially-movable cannula opens the valve.

7. The method of claim 6, wherein the valve includes a compressible sheath.

8. The method of claim 6, wherein the valve includes a duckbill valve.

9. The method of claim 4, wherein the housing further includes a valve disposed on the fixed cannula, the valve having a compressible sheath, and
wherein when the breakaway connector is installed on the needle-free connector, the needle-free connector opens the valve.

10. The method of claim 10, wherein when the breakaway connector is installed on the needle-free connector, the compressible sheath opens the seal.

11. The method of claim 10, wherein when the breakaway connector is installed on the needle-free connector, the fixed cannula opens the seal.

12. A breakaway connector apparatus for attachment to a needle-free connector, comprising:
a breakaway connector including a housing having a fixed cannula, and a breakaway component detachably secured to the housing; and
only one valve disposed within the fixed cannula,
wherein the breakaway component includes a socket configured for engagement with the needle-free connector, such that the needle-free connector imparts a push-out force on the housing,
wherein the breakaway connector includes a breakaway force associated with detaching the breakaway component from the shell, and
wherein a sum of the breakaway force and the push-out force is within a desired range of separation force associated with separating the shell from the needle-free connector.

13. The apparatus of claim 12, wherein the valve includes a compressible sheath.

14. The apparatus of claim 12, wherein the valve includes a duckbill valve.

15. The apparatus of claim 12, further comprising an axially-movable cannula disposed in the fixed cannula.

16. The apparatus of claim 15, wherein the valve includes a compressible sheath.

17. The apparatus of claim 12, wherein the breakaway connector further includes a shell disposed on the housing, and
wherein the shell includes a securing arm.

18. The apparatus of claim 17, wherein the breakaway component includes a ramp and a ramp window, and
wherein the securing arm extends into the ramp window and engages the ramp when the breakaway component is secured to the housing.

19. The apparatus of claim 18, wherein the securing arm is configured to disengage from the ramp when the breakaway force is applied to the breakaway connector.

20. A kit for attaching a breakaway connector device to a needle-free connector that imparts a push-out force, comprising:
a first breakaway connector having a first housing and a first breakaway component, wherein the first breakaway connector provides a first breakaway force associated with separation of the first housing from the first breakaway component; and
a second breakaway connector having a second housing and a second breakaway component, wherein the second breakaway connector provides a second breakaway force associated with separation of the second housing from the second breakaway component, and wherein the second breakaway force is different than the first breakaway force,
wherein each of the first breakaway connector and the second breakaway connector are configured for attachment to the needle-free connector, and
wherein at least one of the first breakaway force and the second breakaway force, when combined with the push-out force imparted by the needle-free fitting, provides a sum that is within a desired range of separation force associated with separating the first housing or the second housing, respectively, from the needle-free fitting.
